(19) United States
(12) Patent Application Publication
(10) Pub. No.: US 2024/0150297 A1
(43) Pub. Date: May 9, 2024

(54) NOVEL COMPOUNDS COMPRISING A NEW CLASS OF TRANSTHYRETIN LIGANDS FOR TREATMENT OF COMMON AGE-RELATED COMORBIDITIES

(71) Applicants: The Trustees of Columbia University in the City of New York, New York, NY (US); Albany College of Pharmacy and Health Sciences in the City of Albany, Albany, NY (US); PETRUKIN, Konstantin, Windsor, NY (US); RACZ, Boglarka, Brooklyn, NY (US); VARADI, Andras, Brooklyn, NY (US); CIOFFI, Christopher, L., Albany, NY (US); MUTHURAMAN, Parthasarathy, Albany, NY (US); RAJA, Arun, Albany, NY (US)

(72) Inventors: Konstantin Petrukhin, New Windsor, NY (US); Boglarka Racz, Brooklyn, NY (US); Andras Varadi, Brooklyn, NY (US); Christopher : Cioffi, Albany, NY (US); Parthasarathy Muthuraman, Albany, NY (US); Arun Raja, Albany, NY (US)

(73) Assignees: The Trustees of Columbia University in the City of New York, New York, NY (US); Albany College of Pharmacy and Health Sciences in the City of Albany, Albany, NY (US)

(21) Appl. No.: 18/264,994

(22) PCT Filed: Feb. 10, 2022
(86) PCT No.: PCT/US2022/015917

§ 371 Date: Aug. 10, 2023

Related U.S. Application Data
(60) Provisional application No. 63/149,124, filed on Feb. 12, 2021.

(51) Int. Cl. C07D 231/38; A61K 31/496; A61P 27/02; C07D 261/14; C07D 275/03; C07D 403/12; C07D 413/12; C07D 417/12

(52) U.S. Cl. CPC C07D 231/38; A61K 31/496; A61P 27/02; C07D 261/14; C07D 275/03; C07D 403/12; C07D 413/12; C07D 417/12

(57) ABSTRACT

The present invention provides a compound having the structure:

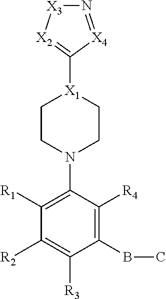

wherein
X₁ is N or CR₅,
wherein R₅ is H, OH, halogen or alkyl;
   X₂, X₃ and X₄ are each independently NH, N, S, O or CR₆, wherein each R₆ is independently H, OH, halogen, alkyl, alkenyl, alkynyl, cycloalkyl, cycloalkylalkyl, haloalkyl, —O—(alkyl), —S—(alkyl), —NH₂, —NH—(alkyl), —N(alkyl)₂ or —CO₂H;
   R₁, R₂, R₃ and R₄ are each independently —H, —F, —Cl, —Br, —I, —NO₂, —CN, —CF₃, —CF₂H, —OCF₃, -(alkyl), -(haloalkyl), -(alkenyl), -(alkynyl), -(aryl), -(heteroaryl), -(cycloalkyl), -(cycloalkylalkyl), -(heteroalkyl), heterocycle, heterocycloalkyl, -(alkylheteroalkyl), -(alkylaryl), —OH, —OAc, —O-(alkyl), —O-(alkenyl), —O-(alkynyl), —O-(aryl), (heteroaryl), —SH, —S-(alkyl), —S-(alkenyl), —S-(alkynyl), —S-(aryl), —S-(heteroaryl), —NH₂, —NH-(alkyl), —NH-(alkenyl), —NH-(alkynyl), —NH-(aryl), —NH-(heteroaryl), —C(O)R₇, —S(O)R₇, —SO₂R₇, —NHSO₂R₇, —OC(O)R₇, —SC(O)R₇, —NHC(O)R₇ or —NHC(S)R₇,
   wherein R₇ is, H, —(alkyl), —OH, —O(alkyl), —NH₂, —NH(alkyl) or —N(alkyl)₂;
   B is absent or present, and when present is

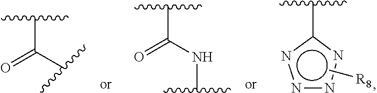

wherein R₈ is H, OH, halogen, alkyl, cycloalkyl,
cycloalkylalkyl, —O-(alkyl), —S-(alkyl), —NH₂, —NH-(alkyl), —N(alkyl)₂ or —CO₂H; and
   C is H, substituted or unsubstituted monocycle, bicycle, heteromonocycle, heterobicycle, aryl, heteroaryl, alkyl, cycloalkyl, cycloalkylalkyl, CO₂H, COOR₉, OH, OR₉, NH₂, NHR₉, NR₉R₁₀, SO₂R₁₁, CH₂NHR₉, CH₂NR₉R₁₀ or CH₂COOR₉,
wherein R₉ and R₁₀ are each independently H, alkyl, cycloalkyl, —C(O)-alkyl, —C(O)-cycloalkyl, —C(O)OH, —C(O)—O-alkyl, —C(O)—O-cycloalkyl, —C(O)NH₂, —C(O)NH(alkyl), —C(O)NH(cycloalkyl), —C(O)N(alkyl)₂, —CH₂NH(alkyl), —CH₂COOH, —SO₂CH₃, —OH, —O(alkyl), —NH₂, —NH(alkyl) or —N(alkyl)
wherein R₁₁ is alkyl, haloalkyl, cycloalkyl, aryl, heteroaryl, NH₂, NH(alkyl), NH(cycloalkyl), NH(heterocycle), NH(aryl), NH(heteroaryl) or NHCOR₁₂,
   wherein R₁₂ is alkyl, haloalkyl, cycloalkyl, heterocycle, aryl or heteroaryl,
      or a pharmaceutically acceptable salt thereof.

### CROSS-REFERENCE TO RELATED APPLICATIONS

This application is a §371 national stage of PCT International Application No. PCT/US2022/015917, filed Feb. 10, 2022, claiming the benefit of U.S. Provisional Application No. 63/149,124, filed Feb. 12, 2021, the contents of each of which are hereby incorporated by reference into the subject application.

This invention was made with government support under R₀₁EY028549 awarded by the National Institutes of Health. The government has certain rights in the invention.

Throughout this application, certain publications are referenced in parentheses. Full citations for these publications may be found immediately preceding the claims. The disclosures of these publications in their entireties are hereby incorporated by reference into this application in order to describe more fully the state of the art to which this invention relates.

### BACKGROUND OF THE INVENTION

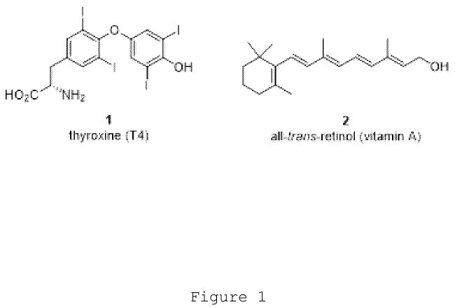

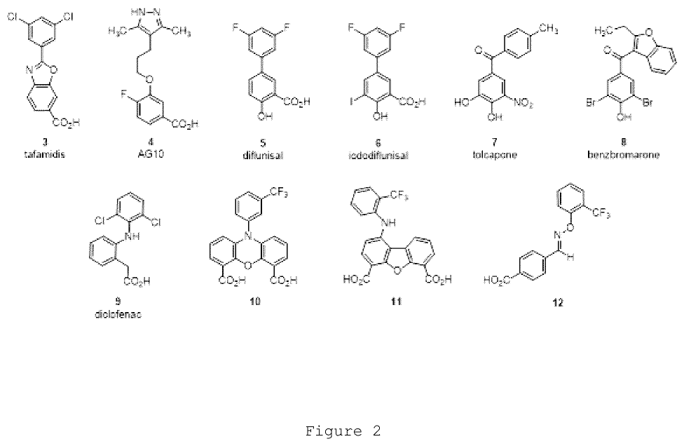

Transthyretin (TTR, thyroxine binding prealbumin) is a 55 kDa homotetramer comprised of four β-sheet-rich, 127-residue polypeptide monomers that is largely synthesized in the liver for secretion into the blood (Vieira, M. & Saraiva 2014). TTR tetramers possess two high-affinity binding sites for the thyroid hormone thyroxine (T4, 1) (FIG. 1). However, less than 1% of circulating TTR carries T4 while another serum protein, thyroxine binding globulin (TBG), functions as its primary transporter in the blood (Vieira, M. & Saraiva 2014). While TTR is not a primary carrier of T4 in the serum, it serves as the major transport protein for the hormone in the central nervous system (CNS) where choroid plexus-derived TTR delivers T4 from the cerebral spinal fluid (CSF) to the choroid plexus and the brain (Kassem, N. A. et al. 2006). Accumulating evidence suggests that TTR may play an auxiliary role in sequestering β-amyloid (Aβ) peptides within the CSF by promoting their clearance from the CNS to the periphery, potentially providing neuroprotective effects against Alzheimer's disease (AD) (Gimeno, A. et al. 2017; Giao, T. et al. 2020; Gales, L. et al. 2005). In systemic circulation, a significant portion of circulating TTR (approximately 50%) forms a macromolecular complex with retinol binding protein 4 (RBP4) associated with all-trans-retinol (vitamin A, 2) (FIG. 1) (Kanai, M. et al. 1968; Hyung, S. J. et al. 2010). This retinol-dependent RBP4-TTR interaction is essential for efficient systemic trafficking of all-trans-retinol as it prevents glomerular filtration of the low molecular weight RBP4-all-trans-retinol complex (Kawaguchi, R. et al. 2015).

The circulating TTR molecule is a homotetramer formed by two dimers (Vieira, M. & Saraiva 2014). To form the homotetrameric structure, two TTR monomers initially associate in a dimer subunit, which further associates with a second dimer subunit. The resulting dimer of dimer architecture presents a tetramer bearing two identical C2 symmetric T4-binding sites located within a central channel of the tetramer and formed at the dimer-dimer interface (Vieira, M. & Saraiva 2014). The TTR dimer-dimer interface is relatively weak and its dissociation is the rate-limiting step in the overall TTR tetramer dissociation process (Sun, X. et al. 2018). The free dimer subunits may subsequently further dissociate into monomers that could potentially proceed to misfold and oligomerize. Oligomerization can eventually lead to aggregation and formation of toxic amyloid fibrils, which underlies the pathophysiology of TTR amyloidosis (ATTR) (Sun, X. et al. 2018).

Autosomal dominant ATTR is a rare and progressive disease that involves severe organ damage due to the extracellular deposition of the aforementioned toxic TTR amyloid fibrils in tissues. The disease typically presents clinically as either TTR amyloid cardiomyopathy (ATTR-CM; can lead to arrhythmias, arterial fibrillation, and biventricular heart failure) (Ruberg, F. L. et al. 2019; Yamamoto, H. & Yokochi, T. 2019) or as peripheral polyneuropathy (ATTR-PN; can cause loss of sensation, tingling, numbness, or pain as well as damage to the autonomic nervous system) (Waddington-Cruz, M. et al. 2019) and can arise from pro-pathogenic monomers with inherited TTR mutations. Non-hereditary ATTR may emerge from wild-type TTR (WT-TTR) monomer misfolding in older individuals (Park, G. Y. et al. 2019). There are at least seventy-seven TTR mutations associated with familial ATTR diseases, and these variants influence amyloidogenicity by either 1) reducing the thermodynamic stability of the TTR tetramer (i.e., the monomers are less likely to associate into a TTR tetramer and are more likely to misfold into an amyloidogenic intermediate), 2) by reducing the kinetic barrier for tetramer dissociation (the TTR tetramer with the variant dissociates at a faster rate than WT-TTR with a concomitant increase in monomer aggregation rate), or 3) by both thermodynamically and kinetically destabilizing the TTR tetramer (Leach, B. I. et al. 2018). The kinetically stable but thermodynamically destabilized variant V3OM (Jesus, C. S. et al. 2016) is predominantly associated with late-onset familial amyloid polyneuropathy (FAP) and is strongly pathogenic. The most common amyloidogenic TTR variant, V1221, (Damrauer, S. M. et al. 2019) presents at a relatively high frequency within the African-American population (approximately 3.4%) and is predominantly associated with familial amyloid cardiomyopathy (FAC). Its pathogenicity is attributed to its ability to kinetically destabilize the TTR tetramer and induce a dissociation rate that is approximately 2-fold faster than WT-TTR (Jiang, X. et al. 2001). The L55P mutation both thermodynamically and kinetically destabilizes tetramer formation and can aggressively promote early-onset ATTR-CM and ATTR-PN (Sousa, M. M. et al. 2002). Conversely, compound heterozygotes carrying a pro-amyloidogenic TTR mutation (e.g., V30M) and a disease-suppressing mutation that hyperstabilizes TTR tetramers, such as T119M or R₁₀₄H (Kamata, M. et al. 2009), are reported to either develop a mild late-onset pathology or be completely protected against ATTR. The T119M variant kinetically stabilizes the TTR tetramer whereas the R₁₀₄H variant provides thermodynamic stability to the quaternary structure. This difference in mechanism of stabilization is crucial as the T119M variant is resistant to tetramer dissociation and aggregation and provides a greater level of protection against TTR aggregation in vitro relative to R₁₀₄H. Lastly, WT-TTR misfolding and aggregation that occurs non-genetically with age is associated with senile systemic amyloidosis (SSA), a late-onset and prevalent form of ATTR that is estimated to affect 10% to 20% of individuals aged 80 years and older.

Currently available FDA-approved approaches for treating ATTR-CM and ATTR-PN include two treatments that reduce circulating TTR levels (the antisense oligonucleotide inotersen (Mathew, V. & Wang, A. K. 2019) and the small interfering RNA (siRNA) patisiran (Hoy, S. M. 2018)) and the small molecule tafamidis (vyndagel and vyndamax, 3) (FIG. 2) that binds to and stabilizes TTR tetramers (Bulawa, C. E. et al. 2012; Coelho, T. et al. 2013; Coelho, T. et al. 2016; Cruz, M. W. 2019; Lamb, Y. N. & Deeks, E. D. 2019; Park, J. et al. 2020). Ligand binding at the T4 sites has been shown to kinetically stabilize TTR tetramers by increasing the dissociative energy barrier of the native tetrameric state. Due to the presence of two additional T4 transport proteins (TGB and albumin), the majority of TTR in circulation is not bound to TTR and the T4 binding sites are largely unoccupied (>99% unoccupied). Thus, drug discovery approaches to identify T4-competitive small molecules capable of kinetically stabilizing TTR tetramers has garnered significant interest as a therapeutic option for treating ATTR. Numerous structurally diverse scaffolds in addition to tafamidis have been reported to bind at the T4 site and stabilize TTR tetramers, and representatives of this class are highlighted in FIG. 2 (compounds 3-12). The two most advanced small molecule TTR tetramer stabilizers to date include the aforementioned FDA-approved 3 and clinically investigated AG10 (4) (Alhamadsheh, M. M. et al. 2011; Miller, M. et al. 2018; Penchala, S. C. et al. 2013). TTR stabilizer 3 has been approved for the treatment of FAP and ATTR-CM. A Phase III study with 441 ATTR-CM patients showed administration of 3 reduced the risk of death by 30% and the rate of cardiovascular-related hospitalizations by 32% compared to placebo controls (Maurer, M. S. et al. 2018). TTR stabilizer 4 was reported to be well-tolerated and demonstrated near-complete stabilization of TTR in a 28-day Phase II proof-of-concept trial with ATTR-CM patients presenting symptomatic chronic heart failure (Judge, D. P. et al. 2019). Phase III clinical trials with 4 for the treatment of ATTR-CM and ATTR-PN are currently ongoing. In addition, the repurposed FDA-approved non-steroidal anti-inflammatory drug (NSAID) diflunisal (5) (Berk, J. L. et al. 2013) and catechol-O-methyl transferase (COMT) inhibitor tolcapone (7) (Sant'Anna, R. et al. 2016) are also reported to exhibit TTR tetramer stabilization activity and have been investigated for clinical efficacy against ATTR-PN.

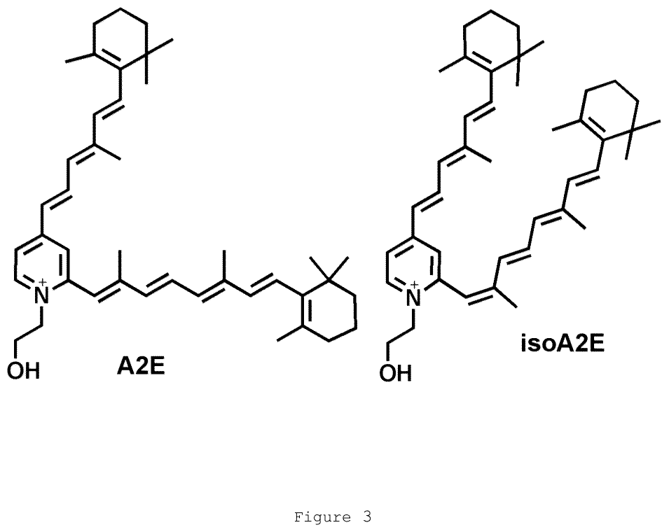

In recent years, the circulating RBP4-TTR-all-trans-retinol transport complex has become a target for pharmacological intervention in ophthalmic diseases associated with enhanced accumulation of cytotoxic lipofuscin bisretinoids, such as A2E, isoA2E, A2-DHP-PE and atRAL di-PE (FIG. 3,4), in the retina. Formation of this transport complex requires that 2 be initially bound to RBP4 (holo-RBP4) as apo-RBP4 poorly associates with TTR (Kawaguchi, R. et al. 2015). Reports indicate that prevention of RBP4-TTR-all-trans-retinol tertiary complex formation can be achieved via selective all-trans-retinol-competitive RBP4 antagonists, which leads to a lowering of serum RBP4 facilitated by rapid glomerular filtration due to its relatively low molecular weight (21 kDa) (Kawaguchi, R. et al. 2015). Evidence suggests that pharmacological reduction of serum RBP4 levels via selective RBP4 antagonists holds therapeutic promise for a variety of diverse indications. For example, it has been hypothesized that RBP4 antagonists may provide a mechanism by which to slow or halt the progression of geographic atrophy in dry age-related macular degeneration (AMD) and Stargardt disease patients by impeding ocular influx of 2 and halting the accumulation of cytotoxic lipofuscin bisretinoids in the retina (Radu, R. A. et al. 2005). Potent and selective RBP4 antagonists disrupt RBP4-TTR-all-trans-retinol tertiary complex formation in vitro and significantly reduce serum RBP4 levels in vivo in rodents, dogs and non-human primates (Cioffi, C. L. et al. 2014; Cioffi, C. L. et al. 2015; Cioffi, C. L. et al. 2019; Racz, B. et al. 2020). Furthermore, chronic oral administration of RBP4 antagonists in Abca4^{-/-} knockout mice, a model of excessive lipofuscinogenesis that recapitulates the Stargardt disease phenotype, led to a reduction in retinal cytotoxic bisretinoid accumulation with an ancillary stabilization of a complement system protein expression in the retinal pigment epithelium (RPE) (Racz, B. et al. 2018; Dobri, N. et al. 2013). Furthermore, additional dosing studies in wild-type BALB/cJ mice revealed that RBP4 antagonist-induced reductions in circulating RBP4 levels correlated with partial reductions in bisretinoid precursor concentrations without disruption of visual cycle kinetics (Racz, B. et al. 2018).

To date, only selective all-trans-retinol-competitive antagonists of RBP4 have been reported to block the formation of a tertiary complex with TTR and lead to a reduction in circulating RBP4 levels in vivo with concomitant inhibition of bisretinoid synthesis in the retina. While selective RBP4 antagonists can be a safe and effective bisretinoid-lowering therapy for a majority of dry AMD and Stargardt disease patients, this class of compounds may potentially be counter-indicated for a fraction of macular degeneration patients who are predisposed to diseases associated with TTR aggregation. Selective RBP4 antagonists would release the unliganded TTR tetramer from the circulating RBP4-TTR-all-trans-retinol transport complex. It has been previously suggested that the RBP4-TTR-all-trans-retinol interaction may stabilize TTR tetramers and the release of a significant pool of unliganded TTR tetramer induced by selective RBP4 antagonists may facilitate TTR amyloid fibril formation in susceptible individuals (Leach, B. I. et al. 2018; Jesus, C. S. et al. 2016) promoting ATTR diseases (Damrauer, S. M. et al. 2019; Jiang, X. et al. 2001; Sousa, M. M. et al. 2002).

This invention describes a novel class of TTR tetramer kinetic stabilizers that selectively bind to TTR tetramers. As such, these compounds have application for the treatment of ATTR-CM, ATTR-PN, FAP, FAC or SSA and other ATTR diseases. Additionally, we here show that these compounds are capable of lowering RBP4 levels so that they also have potential use as therapeutics for the treatment of AMD, dry AMD, Stargardt disease, Best disease, adult vitelliform maculopathy and other conditions characterized by enhanced accumulation of lipofuscin in the retina.

### SUMMARY OF THE INVENTION

The present invention provides a compound having the structure:

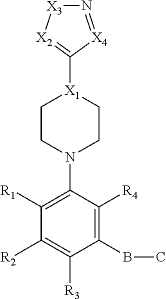

wherein
X₁ is N or CR₅,
   wherein R₅ is H, OH, halogen or alkyl;
X₂, X₃ and X₄ are each independently NH, N, S, O or CR₆,
   wherein each R₆ is independently H, OH, halogen, alkyl, alkenyl, alkynyl, cycloalkyl, cycloalkylalkyl, haloalkyl, —O-(alkyl), —S-(alkyl), —NH₂, —NH-(alkyl), —N(alkyl)₂ or —CO₂H;
R₁, R₂, R₃ and R₄ are each independently —H, —F, —Cl, —Br, —I, —NO₂, —CN, —CF₃, —CF₂H, —OCF₃, -(alkyl), -(haloalkyl), -(alkenyl), -(alkynyl), -(aryl), -(heteroaryl), -(cycloalkyl), -(cycloalkylalkyl), -(heteroalkyl), heterocycle, heterocycloalkyl, -(alkylheteroalkyl), -(alkylaryl), —OH, (heteroaryl), —SH, —S-(alkenyl), —S-(alkynyl), —S-(aryl), —S-(heteroaryl), —NH₂, —NH-(alkyl), —NH-(alkenyl), —NH-SO₂R₇, —NHSO₂R₇, —OC(O)R₇, —SC(O)R₇, —NHC(O)R₇ or —NHC(S)R₇, wherein R₇ is, H, —OH, —O(alkyl), —NH₂, —NH(alkyl) or —N(alkyl)₂;
B is absent or present, and when present is

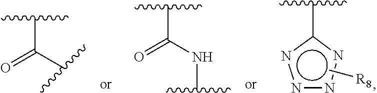

wherein R₈ is H, OH, halogen, alkyl, cycloalkyl, cycloalkylalkyl, —S-(alkyl), —NH₂, —NH-(alkyl), —N(alkyl)₂ or —CO₂H; and C is H, substituted or unsubstituted monocycle, bicycle, heteromonocycle, heterobicycle, aryl, heteroaryl, alkyl, cycloalkyl, cycloalkylalkyl, CO₂H, COOR₅, OH, OR₉, NH₂, NHR₉, NR₉R₁₀, SO₂R₁₁, CH₂NHR₉, CH₂NR₉R₁₀ or CH₂COOR₉, wherein R₉ and R₁₀ are each independently H, alkyl, cycloalkyl, —C(O)-alkyl, —C(O)-cycloalkyl, —C(O)OH, —C(O)—O-cycloalkyl, —C(O)NH₂, C(O)NH(alkyl), —C(O)NH(cycloalkyl), —C(O)N(alkyl)₂, CH₂NH(alkyl), —CH₂COOH, —SO₂CH₃, —OH, —O(alkyl), —NH₂, —NH(alkyl) or —N(alkyl)z,
wherein R₁₁ is alkyl, haloalkyl, cycloalkyl, aryl, heteroaryl, NH₂, NH(alkyl), NH(cycloalkyl), NH(heterocycle), NH(aryl), NH(heteroaryl) or NHCOR₁₂,
   wherein R₁₂ is alkyl, haloalkyl, cycloalkyl, heterocycle, aryl or heteroaryl,
      or a pharmaceutically acceptable salt thereof.

### BRIEF DESCRIPTION OF THE FIGS.

FIG. 1. The thyroid hormone thyroxine (T4) (1) and all-trans-retinol (vitamin A) (2).

FIG. 2. Representative examples of various reported TTR tetramer stabilizer structural classes that bind at the T4 binding site. This sample set of TTR tetramer stabilizers include tafamidis (3), AG10 (4), diflunisal (5), iododiflunisal (6), tolcapone (7), benzbromarone (8), diclofenac (9), N-phenyl phenoxazine 10, dibenzofuran 11, and bisaryloxime ether 12.

FIG. 3. Structure of bisretinoids A2E and isoA2E, cytotoxic components of retinal lipofuscin.

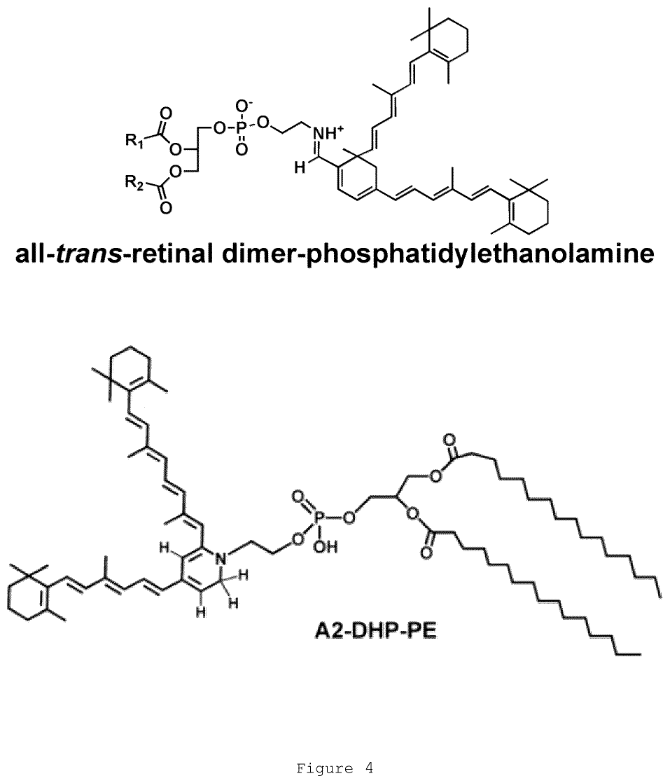

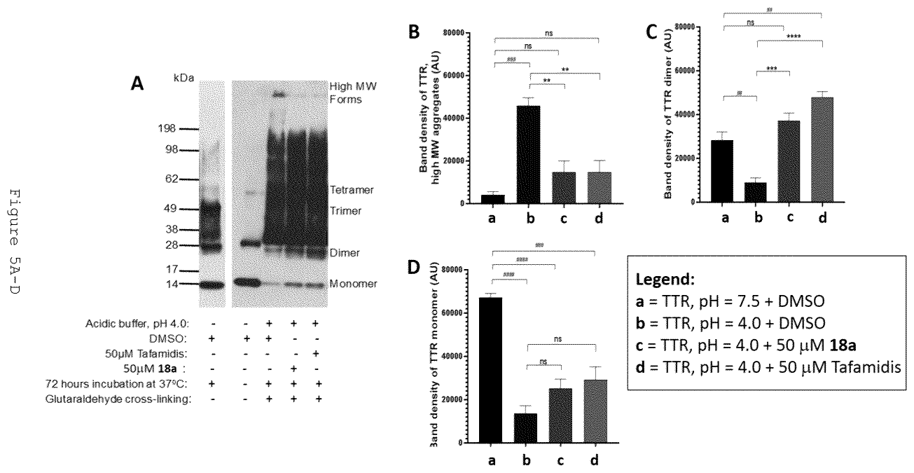

FIG. 4. Structure of bisretinoids atRAL di-PE (all-transretinal dimer-phosphatidyl ethanolamine) and A2-DHP-PE, cytotoxic components of retinal lipofuscin. R₁ and R₂ refer to various fatty acid constituents.

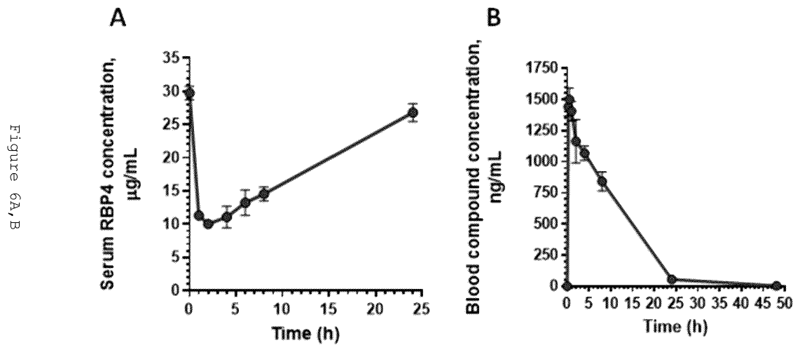

FIG. 5. Analogue 18a reduces the formation of high molecular weight TTR forms in the acid-induced aggregation assay. TTR protein (5 μg) was aggregated by using acetate buffer (pH=4.0) and incubated for 72 h at 37° C. TTR tetramer concentration during the incubation was 9 μM. After incubation in the presence of DMSO, 50 μM 3 (tafamidis), and 50 μM 18a and cross-linking with glutaraldehyde, samples were subjected to SDS-PAGE followed by Western blot analysis of TTR. The representative blot of at least three independent experiments is presented (A). Bar graphs represent pixel volume means±S.D. of the scanned bands on the immunoblots in arbitrary units for TTR high-molecular-weight aggregates (B), dimers (C), and monomers (D). Statistical significance was determined by one-way ANOVA with Holm-Sidak post hoc test; *, p≤0.05**, p≤0.01***, p≤0.001; ****, p≤0.0001 compared to TTR aggregation+DMSO group (pH4.0) *, p=0.05, **, p≤0.01; ***, p≤0.001; ****, p≤0.001 compare to TTR without aggregation group (pH=7.5).

FIG. 6. Pharmacokinetic and pharmacodynamic properties of 18a in mice. (A) Serum RBP4 levels following a single 25 mg/kg oral administration of 18a. (B) Blood compound levels following administration of a single oral 5 mg/kg dose of 18a. Data are presented as means±SD. Three mice per treatment group were used in the PK-PD study.

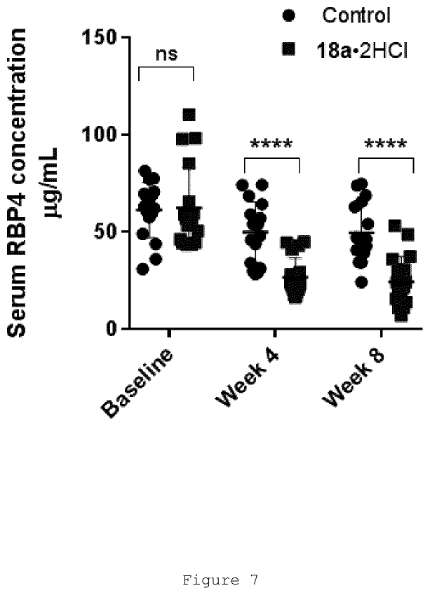

FIG. 7. RBP4 levels in Abca4−/− mice treated with oral 18a·2·HCl for 8 weeks.

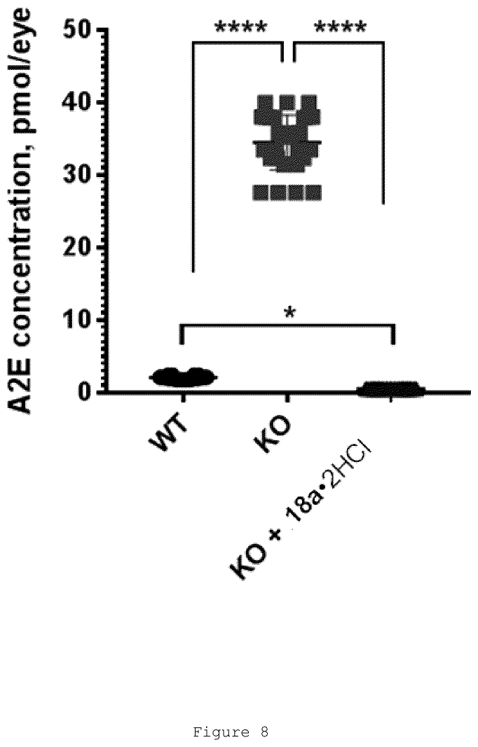

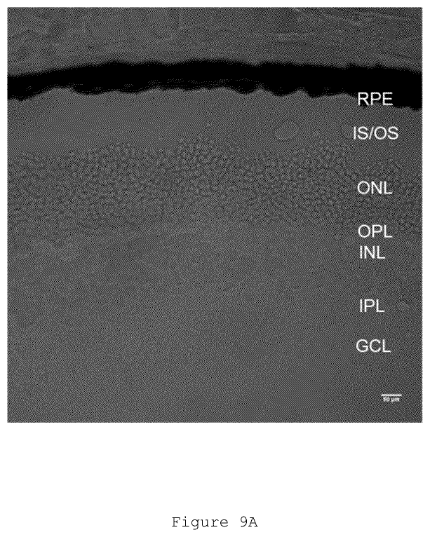

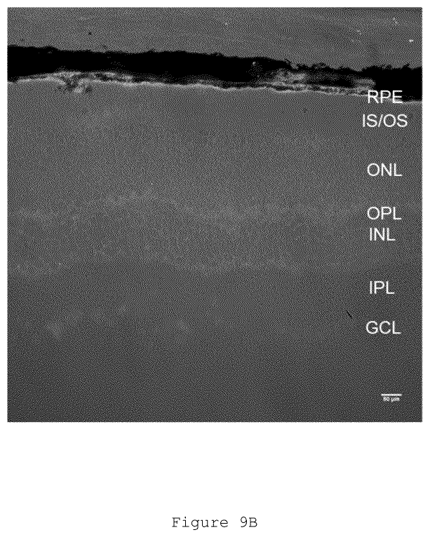

FIG. 8. Bisretinoid reduction efficacy for 18a·2HCl in Abca4−/− mice treated with compound for 8 weeks.

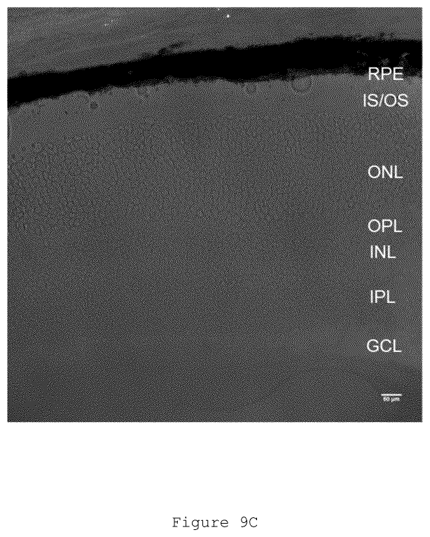

FIG. 9. Lipofuscin autofluorescence in mouse retinal sections. A-C, autofluorescence images from mouse retinal sections prepared from eyes of 12951/SvLmJ untreated mice (A), vehicle-treated 129S-Abca4tm1Ght/J mice (B), and 18a·2HCl-treated 1295-Abca4tm1Ght/J mice (C). 18a·2HCl formulated into a chow was dosed at 27 mg/kg for 6 weeks. The images were captured with confocal microscope under the 40 oil objective using the excitation wavelength of 405 nm (blue, DAPI), 488 (green) nm and emission wavelengths of 420-470 nm (blue, DAPI), 500-600 nm (green). Merged images indicating the localization of peak lipofuscin autofluorescence (green) within retinal layers (blue, DAPI) and retinal organization (DIC) imaged in a differential interference contrast mode. GCL, ganglion cell layer; IPL, inner plexiform layer; INL, inner nuclear layer; OPL, outer plexiform layer; ONL, outer nuclear layer; IS/OS, inner and outer segments of the photoreceptor layer; RPE, retinal pigmented epithelium. Scale bar, 50 μm.

### DETAILED DESCRIPTION OF THE INVENTION

The present invention provides a compound having the structure:

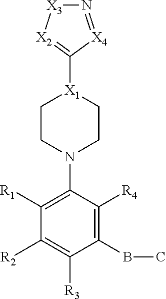

wherein
X₁ is N or CR₅,
   wherein R₅ is H, OH, halogen or alkyl;
X₂, X₃ and X₄ are each independently NH, N, S, O or CR₆,
   wherein each R₆ is independently H, OH, halogen, alkyl, alkenyl, alkynyl, cycloalkyl, cycloalkylalkyl, haloalkyl, —O-(alkyl), —S-(alkyl), —NH₂, —NH-(alkyl), —N(alkyl)₂ or —CO₂H;
R₁, R₂, R₃ and R₄ are each independently —H, —F, —Cl, —Br, —I, —NO₂, —CN, —CF₃, —CF₂H, —OCF₃, -(alkyl), -(haloalkyl), -(alkenyl), -(alkynyl), -(aryl), -(heteroaryl), -(cycloalkyl), -(cycloalkylalkyl), -(heteroalkyl), heterocycle, heterocycloalkyl, -(alkylheteroalkyl), -(alkylaryl), —OH, -(heteroaryl), —SH, —S-(alkyl), —S-(alkenyl), —S-(alkynyl), —S-(aryl), —S-(heteroaryl), —NH₂, —NH-(alkyl), —NH-(alkenyl), —NH-SO₂R₇, —NHSO₂R₇, —OC(O) R₇, —SC(O)R₇, —NHC (O) R₇ or —NHC (S) R₇, wherein R₇ is, H, -(alkyl), —OH, —O(alkyl), —NH₂, —NH(alkyl) or —N(alkyl₂;
B is absent or present, and when present is

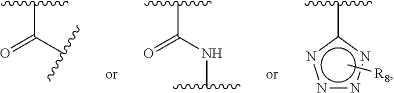

wherein R₈ is H, OH, halogen, alkyl, cycloalkyl, cycloalkylalkyl, —O-(alkyl), —S-(alkyl), —NH₂, —NH-(alkyl), —N(alkyl)₂ or —CO₂H; and
   C is H, substituted or unsubstituted monocycle, bicycle, heteromonocycle, heterobicycle, aryl, heteroaryl, alkyl, cycloalkyl, cycloalkylalkyl, CO₂H, COORS, OH, OR₉, NH₂, NHR₉, NR₉R₁₀, SO_{z}R₁₁, CH₂NHR₉, CH₂NR₉R₁₀ or CH₂COOR₉,
wherein R₉ and R₁₀ are each independently H, alkyl, cycloalkyl, —C(O)-alkyl, —C(O)-cycloalkyl, —C(O)OH, —C(O)—O-alkyl, —C(O)—O-cycloalkyl, —C(O)NH₂, C(O)NH(alkyl), —C(O)NH(cycloalkyl), —C(O)N(alkyl)₂, CH₂NH (alkyl), —CH₂COOH, —SO2CH₃, —OH, —O(alkyl), —NH₂, —NH(alkyl) or —N(alkyl)z, wherein R₁₁ is alkyl, haloalkyl, cycloalkyl, aryl, heteroaryl, NH₂, NH(alkyl), NH(cycloalkyl), NH(heterocycle), NH(aryl), NH(heteroaryl) or NHCOR₁₂,
   wherein R₁₂ is alkyl, haloalkyl, cycloalkyl, heterocycle, aryl or heteroaryl,
      or a pharmaceutically acceptable salt thereof.

In some embodiments, the compound wherein
X₁ is N or CR₅,
   wherein R₅ is H, OH, halogen or alkyl;
X₂, X₃ and X₄ are each independently NH, N, S, O or CR₆, wherein each R₆ is independently H, OH, halogen, alkyl, alkenyl, alkynyl, cycloalkyl, cycloalkylalkyl, haloalkyl, —S-(alkyl), —NH₂, —NH-(alkyl), —N(alkyl)₂ or —CO₂H;
R₁, R₂, R₃ and R₄ are each independently —H, —F, —Cl, —Br, —I, —NO₂, —CN, —CF₃, —CF₂H, —OCF₃, -(alkyl), -(haloalkyl), -(alkenyl), -(alkynyl), -(aryl), -(heteroaryl), -(cycloalkyl), -(cycloalkylalkyl), -(heteroalkyl), heterocycle, heterocycloalkyl, -(alkylheteroalkyl), -(alkylaryl), —OH, —OAc, —O-(alkenyl), —O-(alkynyl), —O-(aryl), —O-(heteroaryl), —SH, —S-(alkenyl), —S-(alkynyl), —S-(aryl), —S-(heteroaryl), —NH₂, —NH-(alkyl), —NH-(alkenyl), —NH-(alkynyl), —NH-(aryl) or —NH-(heteroaryl); B is absent or present, and when present is

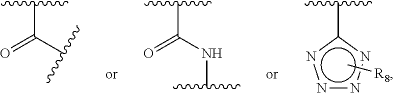

wherein R₈ is H, OH halogen, alkyl, cycloalkyl, cycloalkylalkyl, —O-(alkyl), —S-(alkyl), —NH₂, —NH-(alkyl), —N(alkyl)₂ or —CO₂H; and
   C is H, substituted or unsubstituted monocycle, bicycle, heteromonocycle, heterobicycle, aryl, heteroaryl, alkyl, cycloalkyl, cycloalkylalkyl, CO₂H, COOR S , OH, OR₉, NH₂, NHR₉, NR₉R₁₀, SO₂R₁₁, CH₂NHR₉, CH₂NR₉R₁₀ or CH₂COOR₉,
wherein R₉ and R₁₀ are each independently H, alkyl, cycloalkyl, —C(O)-alkyl, —C(O)-cycloalkyl, —C(O)NH₂, —C(O)—O-alkyl, —C(O)-O-cycloalkyl, —C(O) NH₂, C(O)NH(alkyl), —C(O)NH(cycloalkyl), -—(O)N(alkyl)₂, CH₂NH(alkyl), —CH₂COOH, —SO₂CH₃, —OH, —O(alkyl), —NH₂, —NH(alkyl) or —N(alkyl)₂,
   wherein R₁₁ is alkyl, haloalkyl, cycloalkyl, aryl, heteroaryl, NH₂, NH(alkyl), NH(cycloalkyl), NH(heterocycle), NH(aryl), NH(heteroaryl) or NHCOR₁₂, wherein R₁₂ is alkyl, haloalkyl, cycloalkyl, heterocycle, aryl or heteroaryl, or a pharmaceutically acceptable salt thereof.

In some embodiments, the compound wherein
X₁ is N;
X₂, X₃ and X₄ are each independently NH, N, S, O or CR₆, wherein each R₆ is independently H, halogen, alkyl, alkenyl, alkynyl, haloalkyl, —S-(alkyl), —NH₂, —NH-(alkyl), —N(alkyl)₂ or —CO₂H;
R₁, R₂, R₃ and R₄ are each independently —H, —F, —Cl, —Br, —I, —CN, —CF₃, CF₂H, OCF₃, -(alkyl), -(alkenyl), -(alkyny), -(aryl), -(heteroaryl), -(cycloalkyl), -(cycloalkylalkyl), (heteroalkyl), heterocycle, heterocycloalkyl, -(alkylheteroalkyl), -(alkylaryl), —OH, —OAc, —O-(alkyl), (alkenyl), —O-(alkynyl), —O-(aryl), —O-(heteroaryl), —NH₂, —NH-(alkyl), —NH-(alkenyl), —NH-(alkynyl), —NH-(aryl) or —NH-(heteroaryl); and
B-C is —CO₂H, —CONH₂ or

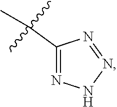 [SMILES: *c1nn[nH]n1]

or a pharmaceutically acceptable salt thereof.

In some embodiments, the compound having the structure:

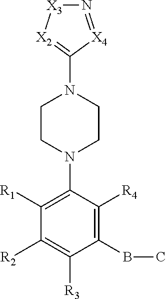

or a pharmaceutically acceptable salt thereof.

In some embodiments, the compound having the structure:

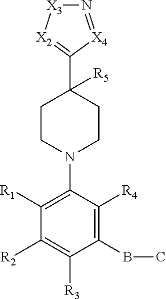

or a pharmaceutically acceptable salt thereof.

In some embodiments, the compound wherein X₃ is NH, and X₂ and X₄ are CR₆.

In some embodiments, the compound wherein X₃ is O, and X₂ and X₄ are CR₆.

In some embodiments, the compound wherein X₃ is S, and X₂ and X₄ are CR₆.

In some embodiments, the compound wherein R₆ is H, OH, alkyl, alkenyl, alkynyl, haloalkyl, —O-(alkyl), —S-(alkyl), —NH₂, —NH-(alkyl), —N(alkyl)₂ or —CO₂H.

In some embodiments, the compound wherein R₆ is alkyl.

In some embodiments, the compound wherein R₆ is methyl.

In some embodiments, the compound wherein R₆ is —CF₃.

In some embodiments, the compound wherein B-C is —CO₂H, —CONH₂ or

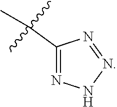 [SMILES: *c1nn[nH]n1]

In some embodiments, the compound wherein B-C is —CO₂H.

In some embodiments, the compound wherein R₁, R₂, R₃ and R₄ are each independently —H, —F, —Cl, —Br, —I, —NO₂, —CN, —CF₃, —CF₂H, —OCF₃, —(alkyl), -(haloalkyl), -(alkenyl), -(alkynyl), —OH, —OAc, —O—(alkyl), —S-(alkyl).

In some embodiments, the compound wherein R₁, R₂, R₃ and R₄ are each independently H, F, Cl, CH₃, CF₃ or OCH₃.

In some embodiments, the compound wherein R₁ is H, F, Cl, CH₃, CF₃ or OCH_{3 .}

In some embodiments, the compound wherein R₂ is H, F, Cl, CH₃, CF₃ or OCH₃.

In some embodiments, the compound wherein R₃ is H, F, Cl, CH₃, CF₃ or OCH₃.

In some embodiments, the compound wherein R₄ is H, F, Cl, CH₃, CF₃ or OCH₃.

In some embodiments, the compound wherein R₁ is H, F, Cl, CH₃, CF₃ or OCH₃, and R₂, R₃ and R₄ are each H.

In some embodiments, the compound wherein R₁ is F, Cl, CH₃, CF₃ or OCH₃, R₃ is CH₃, and R₂ and R₄ are each H.

In some embodiments, the compound wherein R₁ is F and R₂, R₃ and R₄ are each independently H, F, Cl, CH₃, CF₃ or OCH₃.

In some embodiments, the compound wherein R₁ is F and R₂, R₃ and R₄ are each H.

In some embodiments, the compound wherein R₁ is Cl and R₂, R₃ and R₄ are each independently H, F, Cl, CH₃, CF₃ or OCH₃.

In some embodiments, the compound wherein R₁ is Cl and R₂, R₃ and R₄ are each H.

In some embodiments, the compound wherein B-C is —CO₂H, —CONH₂ or

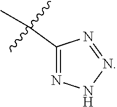 [SMILES: *c1nn[nH]n1]

In some embodiments, the compound wherein R₁ is F or Cl, R₂, R₃ and R₄ are each H, and B-C is —CO₂H.

In some embodiments, the compound wherein R₁ is F or Cl, R₂, R₃ and R₄ are each H, and B-C is —CONH₂.

In some embodiments, the compound wherein R₁ is F or Cl, R₂, R₃ and R₄ are each H, and B-C is

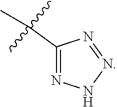 [SMILES: *c1nn[nH]n1]

In some embodiments, the compound having the structure:

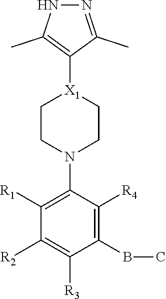

or a pharmaceutically acceptable salt thereof.

In some embodiments, the compound wherein X₁ is N or CR₅.

In some embodiments, the compound wherein B-C is —CO₂H, —CONH₂, or

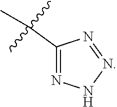 [SMILES: *c1nn[nH]n1]

In some embodiments, the compound having the structure:

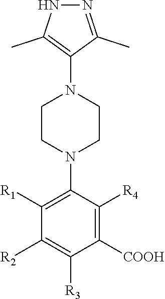

In some embodiments, the compound wherein R₁, R₂, R₃, and R₄ are each independently H, F, Cl, CH₃, CF₃ or OCH_{3.}

In some embodiments, the compound wherein R₁ is H, F, Cl, CH₃, CF₃ or OCH₃.

In some embodiments, the compound wherein R₂ is H, F, Cl, CH₃, CF₃ or OCH_{3.}

In some embodiments, the compound wherein R₃ is H, F, Cl, CH₃, CF₃ or OCH₃.

In some embodiments, the compound wherein R₄ is H, F, Cl, CH₃, CF₃ or OCH₃.

In some embodiments, the compound wherein R₁ is H, F, Cl, CH₃, CF₃ or OCH₃ and R₂, R₃ and R₄ are each H.

In some embodiments, the compound wherein R₁ is F, Cl, CH₃, CF₃ or OCH₃, R₃ is CH₃, and R₂ and R₄ are each H.

In some embodiments, the compound wherein R₁ is F, and R₂, R₃ and R₄ are each H.

In some embodiments, the compound wherein R₁ is Cl, and R₂, R₃ and R₄ are each H.

In some embodiments, the compound has the structure:

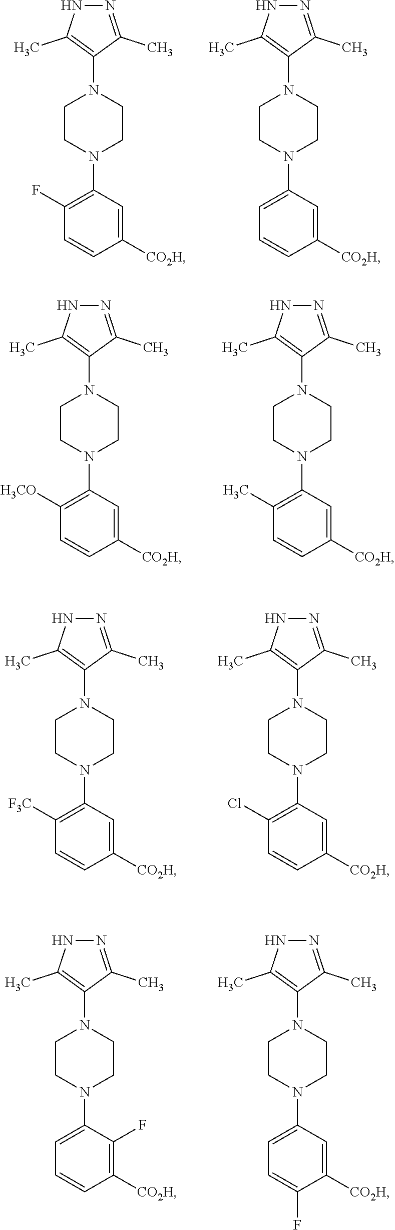

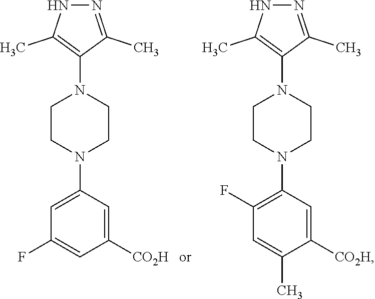

or a pharmaceutically acceptable salt of the compound.

In some embodiments, the compound has the structure:

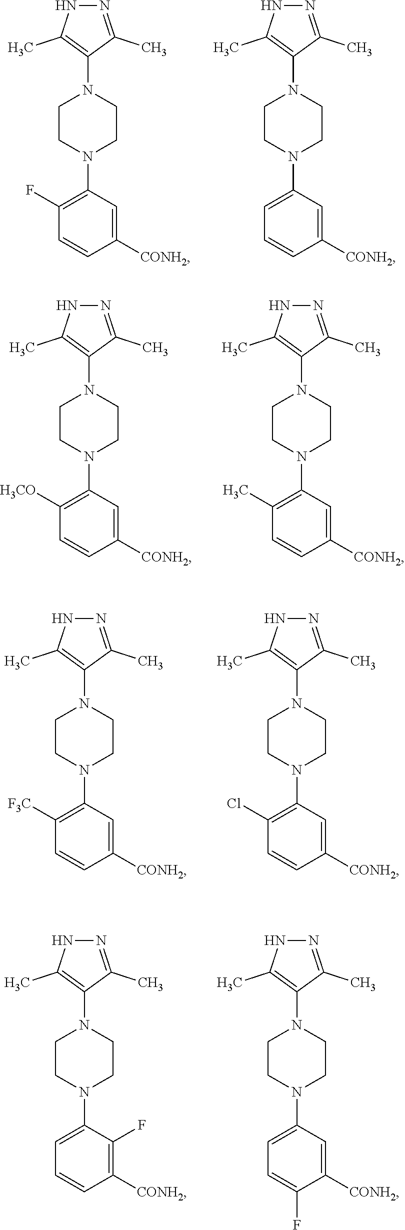

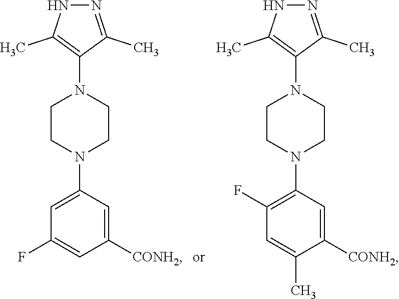

or a pharmaceutically acceptable salt of the compound.

In some embodiments, the compound has the structure:

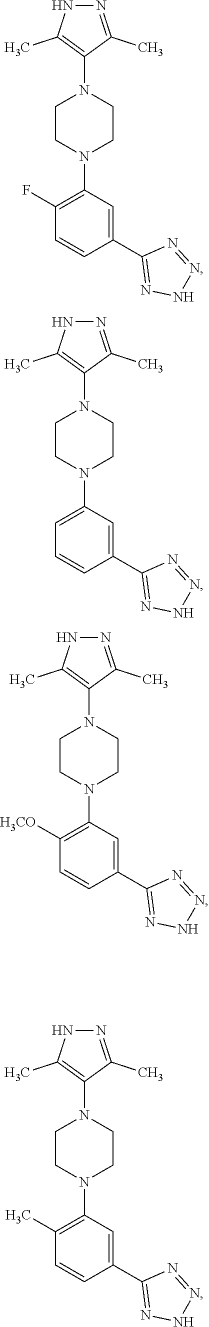 [SMILES: Cc1n[nH]c(C)c1N1CCN(c2cc(-c3nn[nH]n3)ccc2F)CC1 ; Cc1n[nH]c(C)c1N1CCN(c2cccc(-c3nn[nH]n3)c2)CC1 ; COc1ccc(-c2nn[nH]n2)cc1N1CCN(c2c(C)n[nH]c2C)CC1 ; Cc1ccc(-c2nn[nH]n2)cc1N1CCN(c2c(C)n[nH]c2C)CC1]

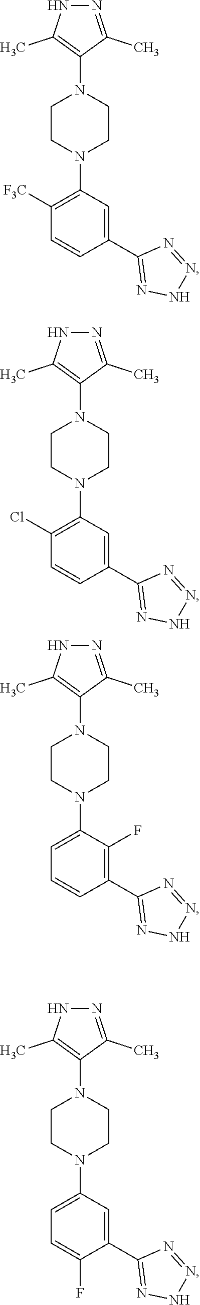 [SMILES: Cc1n[nH]c(C)c1N1CCN(c2cc(-c3nn[nH]n3)ccc2C(F)(F)F)CC1 ; Cc1n[nH]c(C)c1N1CCN(c2cc(-c3nn[nH]n3)ccc2Cl)CC1 ; Cc1n[nH]c(C)c1N1CCN(c2cccc(-c3nn[nH]n3)c2F)CC1 ; Cc1n[nH]c(C)c1N1CCN(c2ccc(F)c(-c3nn[nH]n3)c2)CC1]

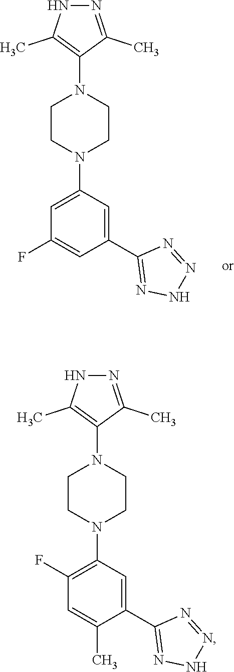 [SMILES: Cc1n[nH]c(C)c1N1CCN(c2cc(F)cc(-c3nn[nH]n3)c2)CC1 ; Cc1cc(F)c(N2CCN(c3c(C)n[nH]c3C)CC2)cc1-c1nn[nH]n1]

or a pharmaceutically acceptable salt of the compound.

In some embodiments, the compound has the structure:

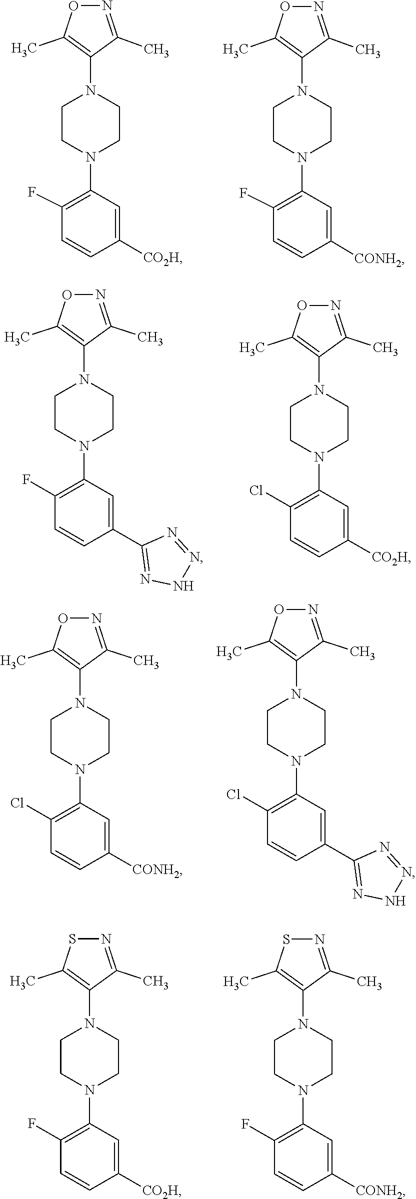

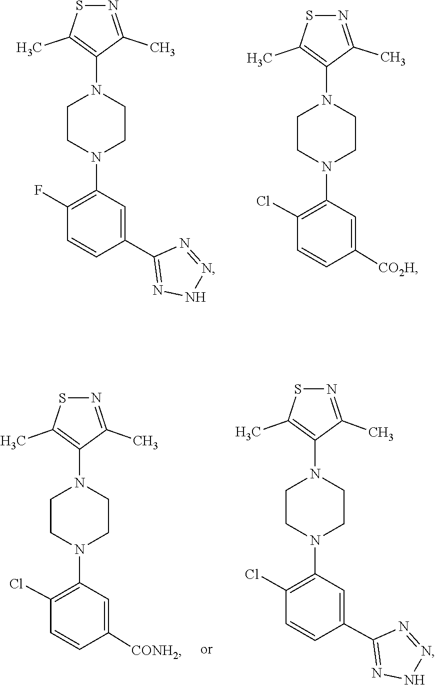

or a pharmaceutically acceptable salt of the compound.

In some embodiments, the compound has the structure:

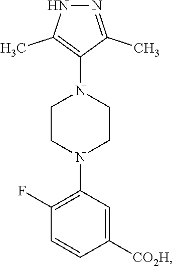

or a pharmaceutically acceptable salt of the compound.

In some embodiments, the compound has the structure:

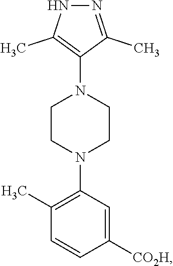

or a pharmaceutically acceptable salt of the compound.

In some embodiments, the compound has the structure:

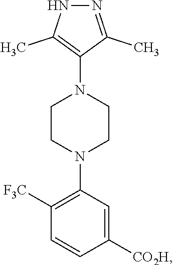

or a pharmaceutically acceptable salt of the compound.

In some embodiments, the compound has the structure:

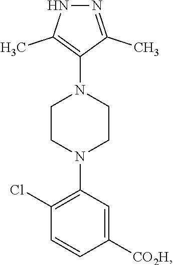

or a pharmaceutically acceptable salt of the compound.

In some embodiments, the compound has the structure:

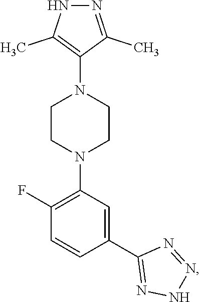 [SMILES: Cc1n[nH]c(C)c1N1CCN(c2cc(-c3nn[nH]n3)ccc2F)CC1]

or a pharmaceutically acceptable salt of the compound.

The present invention provides a pharmaceutical composition comprising the compound of any the present invention and a pharmaceutically acceptable carrier.

The present invention provides a method for stabilizing TTR tetramers in a mammal comprising administering to the mammal an amount of a compound of the present invention or a composition of the present invention effective to stabilize TTR tetramers.

The present invention provides a method of preventing TTR aggregate formation or preventing formation of high molecular weight aggregates in a mammal comprising administering to the mammal an amount of a compound of the present invention or a composition of the present invention effective to prevent TTR aggregate formation or prevent formation of high molecular weight aggregates.

The present invention provides a method for treating a TTR amyloidosis (ATTR) disease, in a mammal afflicted therewith comprising administering to the mammal an effective amount of a compound of the present invention or a composition of the present invention.

In some embodiments of the method, wherein the method is further effective to stabilize TTR tetramers in the mammal.

In some embodiments of the method, wherein the TTR amyloidosis (ATTR) disease is peripheral polyneuropathy (ATTR-PN).

In some embodiments of the method, wherein the TTR amyloidosis (ATTR) disease is TTR amyloid cardiomyopathy (ATTR-CM).

In some embodiments of the method, wherein the TTR amyloidosis (ATTR) disease is late-onset familial amyloid polyneuropathy (FAP).

In some embodiments of the method, wherein the TTR amyloidosis (ATTR) disease is familial amyloid cardiomyopathy (FAC).

In some embodiments of the method, wherein the TTR amyloidosis (ATTR) disease is senile systemic amyloidosis (SSA).

In some embodiments of the method, wherein the TTR amyloidosis (ATTR) disease is characterized by deposition of amyloid aggregates.

The present invention provides a method for treating a disease characterized by excessive lipofuscin accumulation in the retina, in a mammal afflicted therewith comprising administering to the mammal an effective amount of a compound of the present invention or a composition of the present invention.

In some embodiments of the method, wherein the disease is further characterized by bisretinoid-mediated macular degeneration.

In some embodiments of the method, wherein the amount of the compound is effective to lower the serum concentration of RBP4 in the mammal, or wherein the amount of the compound is effective to lower the retinal concentration of a bisretinoid in lipofuscin in the mammal.

In some embodiments of the method, wherein the bisretinoid is A2E.

In some embodiments of the method, wherein the bisretinoid is isoA2E.

In some embodiments of the method, wherein the bisretinoid is A2-DHP-PE.

In some embodiments of the method, wherein the bisretinoid is atRAL di-PE.

In some embodiments of the method, wherein the disease characterized by excessive lipofuscin accumulation in the retina is Age-Related Macular Degeneration.

In some embodiments of the method, wherein the disease characterized by excessive lipofuscin accumulation in the retina is dry (atrophic) Age-Related Macular Degeneration.

In some embodiments of the method, wherein the disease characterized by excessive lipofuscin accumulation in the retina is Stargardt Disease.

In some embodiments of the method, wherein the disease characterized by excessive lipofuscin accumulation in the retina is Best disease.

In some embodiments of the method, wherein the disease characterized by excessive lipofuscin accumulation in the retina is adult vitelliform maculopathy.

In some embodiments of the method, wherein the disease characterized by excessive lipofuscin accumulation in the retina is Stargardt-like macular dystrophy.

The present invention provides a method for treating a disease characterized by a TTR amyloidosis (ATTR) disease, or by excessive lipofuscin accumulation in the retina, or both a TTR amyloidosis (ATTR) disease and a disease characterized by excessive lipofuscin, in a mammal afflicted therewith comprising administering to the mammal an effective amount of a compound of the present invention or a composition of the present invention.

In some embodiments of the method, wherein the amount of the compound is effective to stabilize TTR tetramers in the mammal.

In some embodiments of the method, wherein the amount of the compound is effective to prevent TTR aggregate formation or prevent formation of high molecular weight aggregates.

In some embodiments of the method, wherein the amount of the compound is effective to lower the serum concentration of RBP4 in the mammal, or wherein the amount of the compound is effective to lower the retinal concentration of a bisretinoid in lipofuscin in the mammal.

In some embodiments of the method, wherein the amount of the compound is effective to stabilize TTR tetramers in the mammal and to lower the serum concentration of RBP4 in the mammal.

In some embodiments of the method, wherein the amount of the compound is effective to prevent TTR aggregate formation or prevent formation of high molecular weight aggregates in the mammal and to lower the serum concentration of RBP4 in the mammal.

In some embodiments of the method, wherein the amount of the compound is effective to stabilize TTR tetramers in the mammal and to lower the retinal concentration of a bisretinoid in lipofuscin in the mammal.

In some embodiments of the method, wherein the amount of the compound is effective to prevent TTR aggregate formation or prevent formation of high molecular weight aggregates in the mammal and to lower the retinal concentration of a bisretinoid in lipofuscin in the mammal.

In some embodiments of the method, wherein the TTR amyloidosis (ATTR) disease is peripheral polyneuropathy (ATTR-PN).

In some embodiments of the method, wherein the TTR amyloidosis (ATTR) disease is TTR amyloid cardiomyopathy (ATTR-CM).

In some embodiments of the method, wherein the TTR amyloidosis (ATTR) disease is late-onset familial amyloid polyneuropathy (FAP).

In some embodiments of the method, wherein the TTR amyloidosis (ATTR) disease is familial amyloid cardiomyopathy (FAC).

In some embodiments of the method, wherein the TTR amyloidosis (ATTR) disease is senile systemic amyloidosis (SSA).

In some embodiments of the method, wherein the TTR amyloidosis (ATTR) disease is characterized by deposition of amyloid aggregates.

In some embodiments of the method, wherein the disease is further characterized by bisretinoid-mediated macular degeneration.

In some embodiments of the method, wherein the amount of the compound is effective to lower the serum concentration of RBP4 in the mammal, or wherein the amount of the compound is effective to lower the retinal concentration of a bisretinoid in lipofuscin in the mammal.

In some embodiments of the method, wherein the bisretinoid is A2E.

In some embodiments of the method, wherein the bisretinoid is isoA2E.

In some embodiments of the method, wherein the bisretinoid is A2-DHP-PE.

In some embodiments of the method, wherein the bisretinoid is atRAL di-PE.

In some embodiments of the method, wherein the disease characterized by excessive lipofuscin accumulation in the retina is Age-Related Macular Degeneration.

In some embodiments of the method, wherein the disease characterized by excessive lipofuscin accumulation in the retina is dry (atrophic) Age-Related Macular Degeneration.

In some embodiments of the method, wherein the disease characterized by excessive lipofuscin accumulation in the retina is Stargardt Disease.

In some embodiments of the method, wherein the disease characterized by excessive lipofuscin accumulation in the retina is Best disease.

In some embodiments of the method, wherein the disease characterized by excessive lipofuscin accumulation in the retina is adult vitelliform maculopathy.

In some embodiments of the method, wherein the disease characterized by excessive lipofuscin accumulation in the retina is Stargardt-like macular dystrophy.

The bisretinoid-mediated macular degeneration may comprise the accumulation of lipofuscin deposits in the retinal pigment epithelium.

As used herein, “bisretinoid lipofuscin” is lipofuscin containing a cytotoxic bisretinoid. Cytotoxic bisretinoids include but are not necessarily limited to A2E, isoA2E, atRAL di-PE (all-trans-retinal dimer-phosphatidylethanolamine), and A2-DHP-PE (A2-dihydropyridine-phosphatidylethanolamine) (FIGS. 3-4).

As used herein, “high molecular weight aggregates” refers to all forms of TTR aggregates with molecular weight higher than 198 kilodaltons (kDa).

Transthyretin (TTR) amyloidosis (ATTR) is a neurodegenerative disease and includes, but is not limited to, senile systemic amyloidosis (SSA), peripheral polyneuropathy (ATTR-PN), or cardiomyopathy (ATTR-CM).

In some embodiments, TTR amyloidosis (ATTR) diseases are characterized by the deposition of amyloid aggregates.

In some embodiments, TTR amyloidosis (ATTR) diseases are characterized by the deposition of amyloid aggregates derived from either mutant (TTRm) or wild-type (TTRwt).

In some embodiments, TTR amyloidosis (ATTR) disease is senile systemic amyloidosis (SSA).

In some embodiments, TTR amyloidosis (ATTR) disease is peripheral polyneuropathy (ATTR-PN).

In some embodiments, TTR amyloidosis (ATTR) disease is cardiomyopathy (ATTR-CM).

In some embodiments, the compounds of the present invention exhibit transthyretin (TTR) tetramer kinetic stabilization activity.

In some embodiments, the compounds of the present invention reduce circulating RBP4 levels while simultaneously stabilizing unliganded TTR tetramers released from the holo-RBP4-TTR complex.

In some embodiments, the compounds of the present invention reduce circulating RBP4 levels.

In some embodiments, the compounds of the present invention stabilize unliganded TTR tetramers released from the holo-RBP4-TTR complex.

In some embodiments, the compounds of the present invention or composition of the present invention may be used for the treatment of dry age-related macular degeneration (AMD) and TTR amyloidosis (ATTR) comorbidities.

In some embodiments, the compounds of the present invention or composition of the present invention may be used for the treatment of dry age-related macular degeneration (AMD) and senile systemic amyloidosis (SSA).

In some embodiments, the compounds of the present invention or composition of the present invention may be used for the treatment of dry age-related macular degeneration (AMD) and peripheral polyneuropathy (ATTR-PN).

In some embodiments, the compounds of the present invention or composition of the present invention may be used for the treatment of dry age-related macular degeneration (AMD) and cardiomyopathy (ATTR-CM).

In some embodiments, the compounds of the present invention or composition of the present invention may be used for the treatment of type 2 diabetes.

In some embodiments, the compounds of the present invention or composition of the present invention may be used for the treatment of obesity.

In some embodiments, the compounds of the present invention or composition of the present invention may be used for the treatment of insulin resistance.

In some embodiments, the compounds of the present invention or composition of the present invention may be used for the treatment of cardiovascular disease.

In some embodiments, the compounds of the present invention or composition of the present invention may be used for the treatment of hepatic steatosis.

In some embodiments, the compounds of the present invention or composition of the present invention may be used for the treatment of non-alcoholic fatty liver disease (NAFLD).

In some embodiments, the mammal is a human.

A person skilled in the art may use the techniques disclosed herein to prepare deuterium analogs thereof.

Except where otherwise specified, the structure of a compound of this invention includes an asymmetric carbon atom, it is understood that the compound occurs as a racemate, racemic mixture, scalemic mixtures and isolated single enantiomers. All such isomeric forms of these compounds are expressly included in this invention. Except where otherwise specified, each stereogenic carbon may be of the R or S configuration. It is to be understood accordingly that the isomers arising from such asymmetry (e.g., all enantiomers and diastereomers) are included within the scope of this invention, unless indicated otherwise. Such isomers can be obtained in substantially pure form by classical separation techniques and by stereochemically controlled synthesis, such as those described in “Enantiomers, Racemates and Resolutions” by J. Jacques, A. Collet and S. Wilen, Pub. John Wiley & Sons, NY, 1981. For example, the resolution may be carried out by preparative chromatography on a chiral column.

Except where otherwise specified, the subject invention is intended to include all isotopes of atoms occurring on the compounds disclosed herein. Isotopes include those atoms having the same atomic number but different mass numbers. By way of general example and without limitation, isotopes of hydrogen include tritium and deuterium. Isotopes of carbon include C-13 and C-14.

It will be noted that any notations of a carbon in structures throughout this application, when used without further notation, are intended to represent all isotopes of carbon, such as ¹²C, ¹³C, or ¹⁴C. Furthermore, any compounds containing ¹³C or ¹⁴C may specifically have the structure of any of the compounds disclosed herein.

It will also be noted that any notations of a hydrogen (H) in structures throughout this application, when used without further notation, are intended to represent all isotopes of hydrogen, such as ¹H, ²H (D), or ³H (T) except where otherwise specified. Furthermore, any compounds containing ²H or ³H may specifically have the structure of any of the compounds disclosed herein except where otherwise specified.

Isotopically labeled compounds can generally be prepared by conventional techniques known to those skilled in the art using appropriate isotopically labeled reagents in place of the non-labeled reagents employed.

Deuterium (²H or D) is a stable, non-radioactive isotope of hydrogen and has an atomic weight of 2.0144. Hydrogen atom in a compound naturally occurs as a mixture of the isotopes ¹H (hydrogen or protium), D (²H or deuterium), and T (³H or tritium). The natural abundance of deuterium is 0.0156%. Thus, in a composition comprising molecules of a naturally occurring compound, the level of deuterium at a particular hydrogen atom site in that compound is expected to be 0.0156%. Thus, a composition comprising a compound with a level of deuterium at any site of hydrogen atom in the compound that has been enriched to be greater than its natural abundance of 0.0156% is novel over its naturally occurring counterpart.

The term “substitution”, “substituted” and “substituent” refers to a functional group as described above in which one or more bonds to a hydrogen atom contained therein are replaced by a bond to non-hydrogen or non-carbon atoms, provided that normal valencies are maintained and that the substitution results in a stable compound. Substituted groups also include groups in which one or more bonds to a carbon(s) or hydrogen(s) atom are replaced by one or more bonds, including double or triple bonds, to a heteroatom. Examples of substituent groups include the functional groups described above, and halogens (i.e., F, Cl, Br, and I); alkyl groups, such as methyl, ethyl, n-propyl, isopropryl, n-butyl, tert-butyl, and trifluoromethyl; hydroxyl; alkoxy groups, such as methoxy, ethoxy, n-propoxy, and isopropoxy; aryloxy groups, such as phenoxy; arylalkyloxy, such as benzyloxy (phenylmethoxy) and p-trifluoromethylbenzyloxy (4-trifluoromethylphenylmethoxy); heteroaryloxy groups; sulfonyl groups, such as trifluoromethanesulfonyl, methanesulfonyl, and p-toluenesulfonyl; nitro, nitrosyl; mercapto; sulfanyl groups, such as methylsulfanyl, ethylsulfanyl and propylsulfanyl; cyano; amino groups, such as amino, methylamino, dimethylamino, ethylamino, and diethylamino; and carboxyl. Where multiple substituent moieties are disclosed or claimed, the substituted compound can be independently substituted by one or more of the disclosed or claimed substituent moieties, singly or plurally. By independently substituted, it is meant that the (two or more) substituents can be the same or different.

In the compounds used in the method of the present invention, the substituents may be substituted or unsubstituted, unless specifically defined otherwise.

In the compounds used in the method of the present invention, alkyl, haloalkyl, alkenyl, alkynyl, aryl, heteroaryl, cycloalkyl, cycloalkylalkyl, heteroalkyl, heterocycle, heterocycloalkyl, alkylheteroalkyl, alkylaryl, monocycle, bicycle, heteromonocycle, and heterobicycle groups can be further substituted by replacing one or more hydrogen atoms with alternative non-hydrogen groups. These include, but are not limited to, halo, hydroxy, mercapto, amino, carboxy, cyano and carbamoyl.

It is understood that substituents and substitution patterns on the compounds used in the method of the present invention can be selected by one of ordinary skill in the art to provide compounds that are chemically stable and that can be readily synthesized by techniques known in the art from readily available starting materials. If a substituent is itself substituted with more than one group, it is understood that these multiple groups may be on the same carbon or on different carbons, so long as a stable structure results.

In choosing the compounds used in the method of the present invention, one of ordinary skill in the art will recognize that the various substituents, i.e. R₁, R₂, etc. are to be chosen in conformity with well-known principles of chemical structure connectivity.

As used herein, “alkyl” includes both branched and straight-chain saturated aliphatic hydrocarbon groups having the specified number of carbon atoms and may be unsubstituted or substituted. Thus, C₁-Cₙ as in “C₂-Cₙ alkyl” is defined to include groups having 1, 2, . . . , n-1 or n carbons in a linear or branched arrangement. For example, C₂-C₆, as in “C₁-C₆ alkyl” is defined to include groups having 1, 2, 3, 4, 5, or 6 carbons in a linear or branched arrangement, and specifically includes methyl, ethyl, n-propyl, isopropyl, n-butyl, t-butyl, pentyl, and hexyl. Unless otherwise specified contains one to ten carbons. Alkyl groups can be unsubstituted or substituted with one or more substituents, including but not limited to halogen, alkoxy, alkylthio, trifluoromethyl, difluoromethyl, methoxy, and hydroxyl. “Haloalkyl” includes any alkyl group containing at least one halogen atom.

The term “alkenyl” refers to a non-aromatic hydrocarbon radical, straight or branched, containing at least 1 carbon to carbon double bond, and up to the maximum possible number of non-aromatic carbon-carbon double bonds may be present. Thus, C₂-cₙ alkenyl is defined to include groups having 1, 2 . . . , n-1 or n carbons. For example, “c₂-C₆ alkenyl” means an alkenyl radical having 2, 3, 4, 5, or 6 carbon atoms, and at least 1 carbon-carbon double bond, and up to, for example, 3 carbon-carbon double bonds in the case of a C₆ alkenyl, respectively. Alkenyl groups include ethenyl, propenyl, butenyl and cyclohexenyl. As described above with respect to alkyl, the straight, branched or cyclic portion of the alkenyl group may contain double bonds and may be substituted if a substituted alkenyl group is indicated. An embodiment can be C₂-C₁₂ alkenyl or C₂-C₈ alkenyl.

The term “alkynyl” refers to a hydrocarbon radical straight or branched, containing at least 1 carbon-to-carbon triple bond, and up to the maximum possible number of non-aromatic carbon-carbon triple bonds may be present. Thus, C₂-Cₙ alkynyl is defined to include groups having 1, 2 . . . , n-1 or n carbons. For example, “C₂-C₆ alkynyl” means an alkynyl radical having 2 or 3 carbon atoms, and 1 carbon-carbon triple bond, or having 4 or 5 carbon atoms, and up to 2 carbon-carbon triple bonds, or having 6 carbon atoms, and up to 3 carbon-carbon triple bonds. Alkynyl groups include ethynyl, propynyl and butynyl. As described above with respect to alkyl, the straight or branched portion of the alkynyl group may contain triple bonds and may be substituted if a substituted alkynyl group is indicated.

An embodiment can be a C₂-Cₙ alkynyl. An embodiment can be C₂-C₁₂ alkynyl or C₃-C₈ alkynyl.

As used herein, “aryl” is intended to mean any stable monocyclic, bicyclic, or polycyclic carbon ring of up to 10 atoms in each ring, wherein at least one ring is aromatic, and may be unsubstituted or substituted. Examples of such aryl elements include but are not limited to: phenyl, p-toluenyl (4-methylphenyl), naphthyl, tetrahydro-naphthyl, indanyl, phenanthryl, anthryl or acenaphthyl. In cases where the aryl substituent is bicyclic and one ring is non-aromatic, it is understood that attachment is via the aromatic ring.

The term “heteroaryl”, as used herein, represents a stable monocyclic, bicyclic or polycyclic ring of up to 10 atoms in each ring, wherein at least one ring is aromatic and contains from 1 to 4 heteroatoms selected from the group consisting of O, N and S. Bicyclic aromatic heteroaryl groups include phenyl, pyridine, pyrimidine or pyridazine rings that are (a) fused to a 6-membered aromatic (unsaturated) heterocyclic ring having one nitrogen atom; (b) fused to a 5- or 6-membered aromatic (unsaturated) heterocyclic ring having two nitrogen atoms; (c) fused to a 5-membered aromatic (unsaturated) heterocyclic ring having one nitrogen atom together with either one oxygen or one sulfur atom; or (d) fused to a 5-membered aromatic (unsaturated) heterocyclic ring having one heteroatom selected from O, N or S. Heteroaryl groups within the scope of this definition include but are not limited to: benzimidazolyl, benzofuranyl, benzofurazanyl, benzopyrazolyl, benzotriazolyl, benzothiophenyl, benzoxazolyl, carbazolyl, carbolinyl, cinnolinyl, furanyl, indolinyl, indolyl, indolazinyl, indazolyl, isobenzofuranyl, isoindolyl, isoquinolyl, isothiazolyl, isoxazolyl, naphthpyridinyl, oxadiazolyl, oxazolyl, oxazoline, isoxazoline, oxetanyl, pyranyl, pyrazinyl, pyrazolyl, pyridazinyl, pyridopyridinyl, pyridazinyl, pyridyl, pyrimidyl, pyrrolyl, quinazolinyl, quinolyl, quinoxalinyl, tetrazolyl, tetrazolopyridyl, thiadiazolyl, thiazolyl, thienyl, triazolyl, azetidinyl, aziridinyl, 1,4-dioxanyl, hexahydroazepinyl, dihydrobenzoimidazolyl, dihydrobenzofuranyl, dihydrobenzothiophenyl, dihydrobenzoxazolyl, dihydrofuranyl, dihydroimidazolyl, dihydroindolyl, dihydroisooxazolyl, dihydroisothiazolyl, dihydrooxadiazolyl, dihydrooxazolyl, dihydropyrazinyl, dihydropyrazolyl, dihydropyridinyl, dihydropyrimidinyl, dihydropyrrolyl, dihydroquinolinyl, dihydrotetrazolyl, dihydrothiadiazolyl, dihydrothiazolyl, dihydrothienyl, dihydrotriazolyl, dihydroazetidinyl, methylenedioxybenzoyl, tetrahydrofuranyl, tetrahydrothienyl, acridinyl, carbazolyl, cinnolinyl, quinoxalinyl, pyrrazolyl, indolyl, benzotriazolyl, benzothiazolyl, benzoxazolyl, isoxazolyl, isothiazolyl, furanyl, thienyl, benzothienyl, benzofuranyl, quinolinyl, isoquinolinyl, oxazolyl, isoxazolyl, indolyl, pyrazinyl, pyridazinyl, pyridinyl, pyrimidinyl, pyrrolyl, tetra-hydroquinoline. In cases where the heteroaryl substituent is bicyclic and one ring is non-aromatic or contains no heteroatoms, it is understood that attachment is via the aromatic ring or via the heteroatom containing ring, respectively. If the heteroaryl contains nitrogen atoms, it is understood that the corresponding N-oxides thereof are also encompassed by this definition.

As used herein, “cycloalkyl” includes cyclic rings of alkanes of three to eight total carbon atoms, or any number within this range (i.e., cyclopropyl, cyclobutyl, cyclopentyl, cyclohexyl, cycloheptyl or cyclooctyl). “Cycloalkylalkyl” includes any alkyl group containing at least one cycloalkyl ring.

As used herein, “heteroalkyl” includes both branched and straight-chain saturated aliphatic hydrocarbon groups having at least 1 heteroatom within the chain or branch. “Alkylheteroalkyl” includes any alkyl group containing at least one heteroalkyl group.

The term “heterocycle”, “heterocyclyl” or “heterocyclic” refers to a mono- or poly-cyclic ring system which can be saturated or contains one or more degrees of unsaturation and contains one or more heteroatoms. Preferred heteroatoms include N, O, and/or S, including N-oxides, sulfur oxides, and dioxides. Preferably the ring is three to ten-membered and is either saturated or has one or more degrees of unsaturation. The heterocycle may be unsubstituted or substituted, with multiple degrees of substitution being allowed. Such rings may be optionally fused to one or more of another “heterocyclic” ring(s), heteroaryl ring(s), aryl ring(s), or cycloalkyl ring(s). Examples of heterocycles include, but are not limited to, tetrahydrofuran, pyran, 1,4-dioxane, 1,3-dioxane, piperidine, piperazine, pyrrolidine, morpholine, thiomorpholine, tetrahydrothiopyran, tetrahydrothiophene, 1,3-oxathiolane, and the like.

As used herein, “heterocycloalkyl” is intended to mean a 5- to 10-membered nonaromatic ring containing from 1 to 4 heteroatoms selected from the group consisting of O, N and S, and includes bicyclic groups. “Heterocyclyl” therefore includes, but is not limited to the following: imidazolyl, piperazinyl, piperidinyl, pyrrolidinyl, morpholinyl, thiomorpholinyl, tetrahydropyranyl, dihydropiperidinyl, tetrahydrothiophenyl and the like. If the heterocycle contains nitrogen, it is understood that the corresponding N-oxides thereof are also encompassed by this definition.

The term “alkylaryl” refers to alkyl groups as described above wherein one or more bonds to hydrogen contained therein are replaced by a bond to an aryl group as described above. It is understood that an “alkylaryl” group is connected to a core molecule through a bond from the alkyl group and that the aryl group acts as a substituent on the alkyl group. Examples of arylalkyl moieties include, but are not limited to, benzyl (phenylmethyl), p-trifluoromethylbenzyl (4-trifluoromethylphenylmethyl), 1-phenylethyl, 2-phenylethyl, 3-phenylpropyl, 2-phenylpropyl and the like.

As used herein, “monocycle” includes any stable polycyclic carbon ring of up to 10 atoms and may be unsubstituted or substituted.

Examples of such non-aromatic monocycle elements include but are not limited to: cyclobutyl, cyclopentyl, cyclohexyl, and cycloheptyl. Examples of such aromatic monocycle elements include but are not limited to: phenyl. As used herein, “heteromonocycle” includes any monocycle containing at least one heteroatom.

As used herein, “bicycle” includes any stable polycyclic carbon ring of up to 10 atoms that is fused to a polycyclic carbon ring of up to 10 atoms with each ring being independently unsubstituted or substituted. Examples of such non-aromatic bicycle elements include but are not limited to: decahydronaphthalene. Examples of such aromatic bicycle elements include but are not limited to: naphthalene. As used herein, “heterobicycle” includes any bicycle containing at least one heteroatom.

The compounds used in the method of the present invention may be prepared by techniques well known in organic synthesis and familiar to a practitioner ordinarily skilled in the art. However, these may not be the only means by which to synthesize or obtain the desired compounds.

The compounds used in the method of the present invention may be prepared by techniques described in Vogel's Textbook of Practical Organic Chemistry, A. I. Vogel, A .R. Tatchell, B. S. Furnis, A. J. Hannaford, P. W. G. Smith, (Prentice Hall) 5^{th} Edition (1996), March's Advanced Organic Chemistry: Reactions, Mechanisms, and Structure, Michael B. Smith, Jerry March, (Wiley-Interscience) 5^{th} Edition (2007), and references therein, which are incorporated by reference herein. However, these may not be the only means by which to synthesize or obtain the desired compounds.

The various R groups attached to the aromatic rings of the compounds disclosed herein may be added to the rings by standard procedures, for example those set forth in Advanced Organic Chemistry: Part B: Reactions and Synthesis, Francis Carey and Richard Sundberg, (Springer) 5th ed. Edition. (2007), the content of which is hereby incorporated by reference.

Another aspect of the invention comprises a compound or composition of the present invention as a pharmaceutical composition.

As used herein, the term “pharmaceutically active agent” means any substance or compound suitable for administration to a subject and furnishes biological activity or other direct effect in the treatment, cure, mitigation, diagnosis, or prevention of disease, or affects the structure or any function of the subject. Pharmaceutically active agents include, but are not limited to, substances and compounds described in the Physicians' Desk Reference (PDR Network, LLC; 64th edition; Nov. 15, 2009) and “Approved Drug Products with Therapeutic Equivalence Evaluations” (U.S. Department of Health and Human Services, 30^{th} edition, 2010), which are hereby incorporated by reference. Pharmaceutically active agents which have pendant carboxylic acid groups may be modified in accordance with the present invention using standard esterification reactions and methods readily available and known to those having ordinary skill in the art of chemical synthesis. Where a pharmaceutically active agent does not possess a carboxylic acid group, the ordinarily skilled artisan will be able to design and incorporate a carboxylic acid group into the pharmaceutically active agent where esterification may subsequently be carried out so long as the modification does not interfere with the pharmaceutically active agent's biological activity or effect.

The compounds used in the method of the present invention may be in a salt form. As used herein, a “salt” is a salt of the instant compounds which has been modified by making acid or base salts of the compounds. In the case of compounds used to treat a disease or medical disorder, the salt is pharmaceutically acceptable. Examples of pharmaceutically acceptable salts include, but are not limited to, mineral or organic acid salts of basic residues such as amines; alkali or organic salts of acidic residues such as phenols; alkali or organic salts of acidic residues such as carboxylic acids. The salts can be made using an organic or inorganic acid. Such acid salts are chlorides, bromides, sulfates, nitrates, phosphates, sulfonates, formates, tartrates, maleates, malates, citrates, benzoates, salicylates, ascorbates, and the like. Phenolate salts are the sodium, potassium, or lithium salts, and the like. Carboxylate salts are the sodium, potassium, or lithium salts, and the like. The term “pharmaceutically acceptable salt” in this respect, refers to the relatively non-toxic, inorganic, and organic acid or base addition salts of compounds of the present invention. These salts can be prepared in situ during the final isolation and purification of the compounds of the invention, or by separately reacting a purified compound of the invention in its free base or free acid form with a suitable organic or inorganic acid or base, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like. (See, e.g., Berge et al. (1977) “Pharmaceutical Salts”, J. Pharm. Sci. 66:1-19).

Aa salt or pharmaceutically acceptable salt is contemplated for all compounds disclosed herein.

As used herein, “treating” means preventing, slowing, halting, or reversing the progression of a disease. Treating may also mean improving one or more symptoms of a disease.

The compounds used in the method of the present invention may be administered in various forms, including those detailed herein. The treatment with the compound may be a component of a combination therapy or an adjunct therapy, i.e. the subject or patient in need of the drug is treated or given another drug for the disease in conjunction with one or more of the instant compounds. This combination therapy can be sequential therapy where the patient is treated first with one drug and then the other or the two drugs are given simultaneously. These can be administered independently by the same route or by two or more different routes of administration depending on the dosage forms employed.

As used herein, a “pharmaceutically acceptable carrier” is a pharmaceutically acceptable solvent, suspending agent or vehicle, for delivering the instant compounds to the animal or human. The carrier may be liquid or solid and is selected with the planned manner of administration in mind. Liposomes are also a pharmaceutically acceptable carrier, as are capsules, coatings, and various syringes.

The dosage of the compounds administered in treatment will vary depending upon factors such as the pharmacodynamic characteristics of a specific chemotherapeutic agent and its mode and route of administration; the age, sex, metabolic rate, absorptive efficiency, health and weight of the recipient; the nature and extent of the symptoms; the kind of concurrent treatment being administered; the frequency of treatment with; and the desired therapeutic effect.

A dosage unit of the compounds used in the method of the present invention may comprise a single compound or mixtures thereof with additional agents. The compounds can be administered in oral dosage forms as tablets, capsules, pills, powders, granules, elixirs, tinctures, suspensions, syrups, and emulsions. The compounds may also be administered in intravenous (bolus or infusion), intraperitoneal, subcutaneous, or intramuscular form, or introduced directly, e.g. by injection, topical application, or other methods, into or onto a site of disease, all using dosage forms well known to those of ordinary skill in the pharmaceutical arts.

The compounds used in the method of the present invention can be administered in admixture with suitable pharmaceutical diluents, extenders, excipients, or carriers (collectively referred to herein as a pharmaceutically acceptable carrier) suitably selected with respect to the intended form of administration and as consistent with conventional pharmaceutical practices. The unit will be in a form suitable for oral, rectal, topical, intravenous, or direct injection or parenteral administration. The compounds can be administered alone or mixed with a pharmaceutically acceptable carrier. This carrier can be a solid or liquid, and the type of carrier is generally chosen based on the type of administration being used. The active agent can be co-administered in the form of a tablet or capsule, liposome, as an agglomerated powder or in a liquid form. Examples of suitable solid carriers include lactose, sucrose, gelatin, and agar. Capsule or tablets can be easily formulated and can be made easy to swallow or chew; other solid forms include granules, and bulk powders. Tablets may contain suitable binders, lubricants, diluents, disintegrating agents, coloring agents, flavoring agents, flow-inducing agents, and melting agents. Examples of suitable liquid dosage forms include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules. Such liquid dosage forms may contain, for example, suitable solvents, preservatives, emulsifying agents, suspending agents, diluents, sweeteners, thickeners, and melting agents. Oral dosage forms optionally contain flavoring and coloring agents. Parenteral and intravenous forms may also include minerals and other materials to make them compatible with the type of injection or delivery system chosen.

Techniques and compositions for making dosage forms useful in the present invention are described in the following references: 7 Modern Pharmaceutics, Chapters 9 and 10 (Banker & Rhodes, Editors, 1979); Pharmaceutical Dosage Forms: Tablets (Lieberman et al. 1981); Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol. 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modem Pharmaceutics Drugs and the Pharmaceutical Sciences, Vol 40 (Gilbert S. Banker, Christopher T. Rhodes, Eds.). All of the aforementioned publications are incorporated by reference herein.

Tablets may contain suitable binders, lubricants, disintegrating agents, coloring agents, flavoring agents, flow-inducing agents, and melting agents. For instance, for oral administration in the dosage unit form of a tablet or capsule, the active drug component can be combined with an oral, non-toxic, pharmaceutically acceptable, inert carrier such as lactose, gelatin, agar, starch, sucrose, glucose, methyl cellulose, magnesium stearate, dicalcium phosphate, calcium sulfate, mannitol, sorbitol and the like. Suitable binders include starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. Disintegrators include, without limitation, starch, methyl cellulose, agar, bentonite, xanthan gum, and the like.

The compounds used in the method of the present invention may also be administered in the form of liposome delivery systems, such as small unilamellar vesicles, large unilamellar vesicles, and multilamellar vesicles. Liposomes can be formed from a variety of phospholipids, such as cholesterol, stearylamine, or phosphatidylcholines. The compounds may be administered as components of tissue-targeted emulsions.

The compounds used in the method of the present invention may also be coupled to soluble polymers as targetable drug carriers or as a prodrug. Such polymers include polyvinylpyrrolidone, pyran copolymer, polyhydroxylpropylmethacrylamide-phenol, polyhydroxyethylasparta-midephenol, or polyethyleneoxide-polylysine substituted with palmitoyl residues. Furthermore, the compounds may be coupled to a class of biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacylates, and crosslinked or amphipathic block copolymers of hydrogels.

Gelatin capsules may contain the active ingredient compounds and powdered carriers, such as lactose, starch, cellulose derivatives, magnesium stearate, stearic acid, and the like. Similar diluents can be used to make compressed tablets. Both tablets and capsules can be manufactured as immediate release products or as sustained release products to provide for continuous release of medication over a period of hours. Compressed tablets can be sugar coated or film coated to mask any unpleasant taste and protect the tablet from the atmosphere, or enteric coated for selective disintegration in the gastrointestinal tract.

For oral administration in liquid dosage form, the oral drug components are combined with any oral, non-toxic, pharmaceutically acceptable inert carrier such as ethanol, glycerol, water, and the like. Examples of suitable liquid dosage forms include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules. Such liquid dosage forms may contain, for example, suitable solvents, preservatives, emulsifying agents, suspending agents, diluents, sweeteners, thickeners, and melting agents.

Liquid dosage forms for oral administration can contain coloring and flavoring to increase patient acceptance. In general, water, a suitable oil, saline, aqueous dextrose (glucose), and related sugar solutions and glycols such as propylene glycol or polyethylene glycols are suitable carriers for parenteral solutions. Solutions for parenteral administration preferably contain a water-soluble salt of the active ingredient, suitable stabilizing agents, and if necessary, buffer substances. Antioxidizing agents such as sodium bisulfite, sodium sulfite, or ascorbic acid, either alone or combined, are suitable stabilizing agents. Also used are citric acid and its salts and sodium EDTA. In addition, parenteral solutions can contain preservatives, such as benzalkonium chloride, methyl- or propyl-paraben, and chlorobutanol. Suitable pharmaceutical carriers are described in Remington's Pharmaceutical Sciences, 17th ed., 1989, a standard reference text in this field.

The compounds used in the method of the present invention may also be administered in intranasal form via use of suitable intranasal vehicles, or via transdermal routes, using those forms of transdermal skin patches well known to those of ordinary skill in that art. To be administered in the form of a transdermal delivery system, the dosage administration will generally be continuous rather than intermittent throughout the dosage regimen.

Parenteral and intravenous forms may also include minerals and other materials to make them compatible with the type of injection or delivery system chosen.

Each embodiment disclosed herein is contemplated as being applicable to each of the other disclosed embodiments. Thus, all combinations of the various elements described herein are within the scope of the invention. Any of the disclosed generic or specific compounds may be applicable to any of the disclosed compositions, processes, or methods.

This invention will be better understood by reference to the Experimental Details which follow, but those skilled in the art will readily appreciate that the specific experiments detailed are only illustrative of the invention as described more fully in the claims, which follow thereafter.

### Experimental Details

General Chemistry. All reactions were performed under a dry atmosphere of nitrogen unless otherwise specified. Indicated reaction temperatures refer to the reaction bath, while room temperature (rt) is noted as 25° C. Commercial grade reagents and anhydrous solvents were used as received from vendors and no attempts were made to purify or dry these components further. Removal of solvents under reduced pressure was accomplished with a Buchi rotary evaporator at approximately 28 mm Hg pressure using a Teflon-linked KNF vacuum pump. Thin layer chromatography was performed using 1″×3″ AnalTech No. 02521 silica gel plates with fluorescent indicator. Visualization of TLC plates was made by observation with either short wave UV light (254 nm lamp), 10% phosphomolybdic acid in ethanol or in iodine vapors. Preparative thin layer chromatography was performed using Analtech, 20×20 cm, 1000 micron preparative TLC plates. Flash column chromatography was carried out using a Teledyne Isco CombiFlash Companion Unit and a Biotage® Selekt System with Teledyne Isco RediSep Rf and Biotage Sfar silica gel columns. If needed, products were purified by reverse phase chromatography, using a Teledyne Isco CombiFlash Companion Unit and a Biotage® Selekt System with a RediSep Gold C18 reverse phase column. Proton NMR spectra were obtained on a 400 MHz Varian nuclear magnetic resonance spectrometer. Chemical shifts (δ) are reported in parts per million (ppm) and coupling constant (J) values are given in Hz, with the following spectral pattern designations: s, singlet; d, doublet; t, triplet, q, quartet; quint, quintet; m, multiplet; dd, doublet of doublets; dt, doublet of triplets; dq; doublet of quartets; br, broad signal. Tetramethylsilane was used as an internal reference. Peak listing, multiplicity designations, and coupling constant calculations were conducted using Mnova v.14 software (Mestrelab Research). Carbon NMR spectra were obtained on a 500 MHz Bruker AV III nuclear magnetic resonance spectrometer and tetramethylsilane was used as an internal reference. Fluorine NMR spectra were obtained on a 400 MHz Bruker AV III nuclear magnetic resonance spectrometer. Any melting points provided are uncorrected and were obtained using a Stanford Research Systems OptiMelt melting point apparatus (MPA100) with an automated melting point system. Mass spectroscopic analyses were performed using ESI ionization on a Waters AQUITY UPLC MS triple quadrapole mass spectrometer. High pressure liquid chromatography (HPLC) purity analysis was performed using a Waters Breeze2 HPLC system with a binary solvent system A and B using a gradient elusion [A, H₂O with 0.1% formic acid; B, CH₃CN with 0.1% formic acid] and flow rate=0.5 mL/min, with UV detection at 254 nm (system equipped with a photodiode array (PDA) detector). An ACQUITY UPLC BEH C18 column, 130 Å, 1.7 μm, 2.1 mm×50 mm was used. High resolution mass spectrometry (HRMS) analysis was performed using an Agilent 6530 Accurate-Mass Q-TOF. All final compounds tested for in vitro and in vivo biological testing were purified to ≥95% purity, and these purity levels were measured by both ¹H NMR and HPLC.

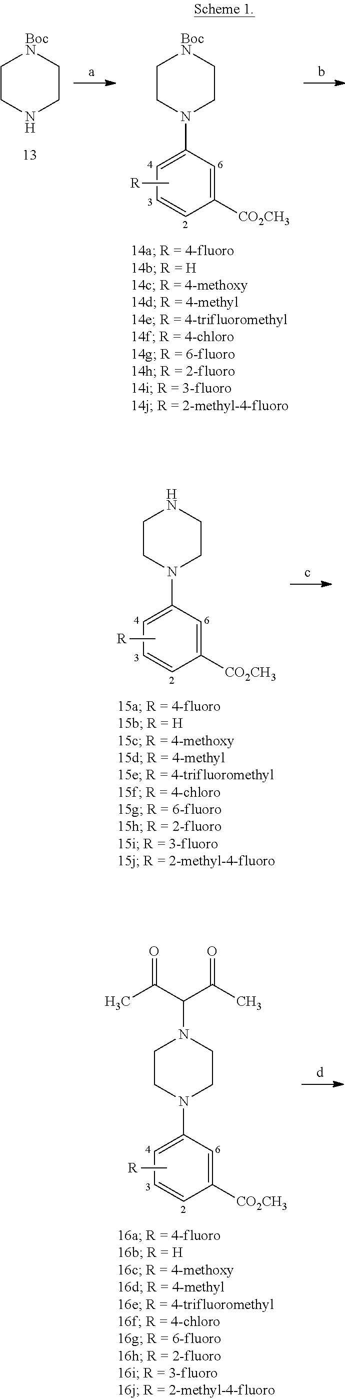

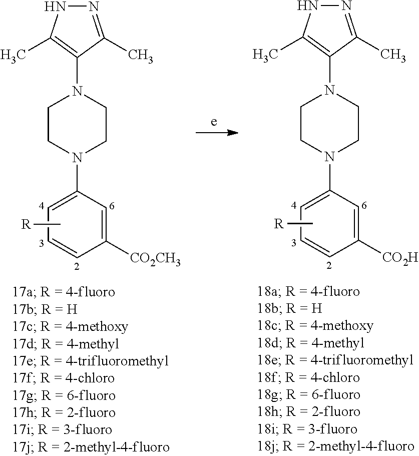

Reagents and conditions: (a) substituted methyl bromobenzoate, XPhos, Pd2(dba)₂, Cs₂CO₃, 1,4-dioxane, reflux, 16 h; (b) TFA, CH₂Cl₂, 0° C. to rt, 16 h; (c) 3-chloropentane-2,4-dione, i-Pr₂NEt, DMF, 0° C. to rt, 16 h; (d) N₂H₂.H₂O (64-65% in H₂O), CH₃OH, rt, 1 h; (e) (i) LiOH, CH₃ OH, THF, H₂O, rt, 16 h; (ii) neutralization to pH=7 with 2 N aqueous HCl.

### Example 1

3-(4-(3,5-Dimethyl-1H-pyrazol-4-yl)piperazin-1-yl)-4-fluorobenzoic Acid 18a. Step A: A mixture of tert-butyl piperazine-1-carboxylate 13 (2.00 g, 10.7 mmol) and methyl 3-bromo-4-fluorobenzoate (2.25 g, 9.67 mmol) in anhydrous 1,4-dioxane (50 mL) was degassed with N₂ for 5 min. Cs₂CO₃ (10.0 g, 32.2 mmol), X-Phos (0.600 g, 1.29 mmol) and Pd₂(dba)₃ (0.491 g, 0.53 mmol) were then added and the mixture was stirred reflux for 16 h under an atmosphere of N₂. The mixture was allowed to cool to rt and then concentrated under reduced pressure. The resulting residue was chromatographed over silica gel (0-30% EtOAc in hexanes) to give tert-butyl 4-(2-fluoro-5-(methoxycarbonyl)phenyl)piperazine-1-carboxylate 14a as a brown oil (3.0 g, 83%). The material was used as is in the next step: ESI MS m/z 339 [M+H]⁺.

Step B: To a 0° C. cooled solution of tert-butyl 4-(2-fluoro-5-(methoxycarbonyl)phenyl)piperazine-1-carboxylate 14a (3.00 g, 8.87 mmol) in CH₂Cl₂ (30 mL) was added TFA (6.7 mL, 88.7 mmol) and the resulting solution was stirred at rt for 16 h while gradually warming to rt. The mixture was then concentrated under reduced pressure and diluted with H₂O (30 mL), basified with saturated aqueous NaHCO₃ solution (50 mL), and extracted with EtOAc (3×50 mL). The combined organic extracts were washed with brine (50 mL), dried over Na₂SO₄, filtered, and concentrated under reduced pressure.

The crude material was triturated with Et₂O and filtered to give pure methyl 4-fluoro-3-(piperazin-1-yl)benzoate 15a as a white solid (1.20 g, 60%): ¹H NMR (400 MHz, CDCl₃) δ9.85 (br, 1H), 7.72-7.69 (m, 1H), 7.63-7.60 (m, 1H), 7.21-7.04 (m, 1H), 3.87 (s, 3H), 3.35 (s, 8H); ESI MS m/z 239 [M+H]⁺.

Step C: To a 0° C. cooled solution of methyl 4-fluoro-3-(piperazin-1-yl)benzoate 15a (1.20 g, 5.02 mmol) in anhydrous DMF (10 mL) were added i-Pr₂NEt (0.9 mL, 5.02 mmol) and 3-chloropentane-2,4-dione (0.672 g, 5.02 mmol) simultaneously and the resulting solution was stirred for 16 h under N₂ atmosphere while gradually warming to rt. The mixture was then diluted with H₂O (50 mL) and extracted with EtOAc (3×50 mL). The combined organic extracts were washed with brine, dried over Na₂SO₄, and concentrated under reduced pressure. The resulting residue was chromatographed over silica gel (0-50% EtOAc in hexanes) to give methyl 3-(4-(2,4-dioxopentan-3-yl)piperazin-1-yl)-4-fluorobenzoate 16a as a brown oil (0.600 g, 35%): ¹H NMR (400 MHz, CDCl₃) δ7.64-7.62 (m, 2H), 7.06-7.01 (m, 1H), 3.86 (s, 1H), 3.31-3.09 (m, 4H), 3.09-3.05 (m, 4H), 2.27 (s, 1H), 2.24 (s, 6H); ESI MS m/z 337 [M+H]⁺.

Step D: To a solution of methyl 3-(4-(2,4-dioxopentan-3-yl)piperazin-1-yl)-4-fluorobenzoate 16a (0.500 g, 1.48 mmol) in CH₃OH (10 mL) was added N₂H₂.H₂O (0.2 mL, 2.67 mmol, 64-65% solution in H₂O) and the resulting mixture stirred at rt for 1 h. The mixture was then concentrated under reduced pressure and the resulting residue was chromatographed over silica gel (0-50% EtOAc in hexanes) to give methyl 3-(4-(3,5-dimethyl-1H-pyrazol-4-yl)piperazin-1-171)-4-fluorobenzoate 17a as a brown solid (0.420 g, 85%): ¹H NMR (400 MHz, CDCl₃) δ7.67-7.63 (m, 2H), 7.04-7.01 (m, 1H), 3.86 (s, 1H), 3.16-3.15 (m, 4H), 3.15-3.11 (m, 4H), 2.24 (s, 6H) ; ESI MS m/z 333 [M+H]⁺.

Step E: To a solution of methyl 3-(4-(3,5-dimethyl-1H-pyrazol-4-yl)piperazin-1-yl)-4-fluorobenzoate 17a (0.420 g, 1.26 mmol) in CH₃OH (4 mL), THF (4 mL) and H₂O (2 mL) was added LiOH (91 mg, 3.79 mmol). The reaction mixture was stirred at rt for 16 h and was concentrated under reduced pressure. The aqueous layer was then diluted with H₂O (30 mL) and neutralized to pH=7 with 2 N aqueous HCl. The aqueous mixture was extracted with EtOAc (3×50 mL) and the combined organic solution was washed with brine, dried over Na₂SO₄, and concentrated under reduced pressure. The crude residue was chromatographed over silica gel (0-10% CH₃OH in CH₂Cl₂) to give 3-(4-(3,5-dimethyl-1H-pyrazol-4-yl)piperazin-1-yl)-4-fluorobenzoic acid 18a as a white solid (0.390 g, 97%): melting point=220−222° C.; ¹H NMR (400 MHz, DMSO-d6) δ7.53-7.55 (m, 2H, H₁ and H₂), 7.23-7.18 (dt, J=12 Hz, 3.2 Hz, 1H, H₃), 3.04 (m, 4H, H4), 3.01 (m, 4H, H₅), 2.10 (s, 6H, H₆); ¹³C NMR (500 MHz, DMSO-d₆) δ128.73, 121.71, 124.10, 124.03, 120.25, 120.21, 116.39, 116.22; ¹⁹F NMR (400 MHz, DMSO-d₆) δ-116.00 (s, F); ESI MS m/z 319 [M+H]⁺; HRMS (ESI⁺) C₁₆H₁₉FN₄O₂ calculated [M+H]⁺=319.157, observed [M+H]⁺=319.1562; combustion analysis (% CHN): calcd for C₁₆H₁₉FN₄O₂·0.5 H₂O 0.5 HCl: % C=55.61; % H=5.98; % N=16.21; found: % C=55.88; % H=5.74; % N=15.97; HPLC>99% (AUC), t_{R}=11.5 min.

### Example 2

3-(4-(3,5-Dimethyl-1H-pyrazol-4-yl)piperazin-1-yl)benzoic Acid 18b. Compound 18b was prepared from tert-butyl piperazine-1-carboxylate 13 and methyl 3-bromobenzoate according to a similar procedure described for the synthesis of 18a: ¹H NMR (400 MHz, DMSO-d₆) δ7.48 (s, 1H), 7.36 (m, 2H), 7.23 (m, 1H), 3.23 (m, 4H), 3.04 (m, 4H), 2.14 (s, 6H); ESI MS m/z 301 [M+H]⁺; HPLC>99% (AUC), t_{R}=10.7 min.

### Example 3

3-(4-(3,5-Dimethyl-1H-pyrazol-4-yl)piperazin-1-yl)-4-methoxybenzoic Acid 18c. Compound 18c was prepared from tert-butyl piperazine-1-carboxylate 13 and methyl 3-bromo-4-methoxybenzoate according to a similar procedure described for the synthesis of 18a: ¹H NMR (400 MHz, DMSO-d₆) δ7.58 (dd, J=8.4 Hz, 2.0 Hz, 1H), 7.44 (d, J=2.0 Hz, 1H), 7.01 (d, J=8.4 Hz, 1H), 3.83 (s, 3H), 2.99 (s, 8H), 2.08 (s, 6H); ESI MS m/z 331 [M+H]⁺; HPLC 94.6% (AUC), t_{R}=10.2 min.

### Example 4

3-(4-(3,5-Dimethyl-1H-pyrazol-4-yl)piperazin-1-yl)-4-methylbenzoic Acid 18d. Compound 18d was prepared from tert-butyl piperazine-1-carboxylate 13 and methyl 3-bromo-4-methylbenzoate according to a similar procedure described for the synthesis of 18a: ¹H NMR (400 MHz, DMSO-d₆) δ7.57 (d, J=1.2 Hz, 1H), 7.53 (d, J=8.0 Hz, 1H), 7.27 (d, J=8.0 Hz, 1H), 3.01 (m, 4H), 2.90 (m, 4H), 2.46 (s, 3H), 2.12 (s, 6H); ESI MS m/z 315 [M+H]⁺; HPLC>99% (AUC), t_{R}=11.2 min.

### Example 5

3-(4-(3,5-Dimethyl-1H-pyrazol-4-yl)piperazin-1-yl)-4-(trifluoromethyl)benzoic Acid 18e. Compound 18e was prepared from tert-butyl piperazine-l-carboxylate 13 and methyl 3-bromo-4-(trifluoromethyl)benzoate according to a similar procedure described for the synthesis of 18a: ¹H NMR (400 MHz, DMSO-d₆) δ7.99 (s, 1H), 7.84 (d, J=8.4 Hz, 1H), 7.78 (d, J=8.4 Hz, 1H), 2.97 (m, 4H), 2.94 (m, 4H), 2.11 (s, 6H); ESI MS m/z 369 [M+H]⁺; HPLC 98.7% (AUC), t_{R}=11.9 min.

### Example 6

4-Chloro-3-(4-(3,5-dimethyl-1H-pyrazol-4-yl)piperazin-1-yl)benzoic Acid 18f. Compound 18f was prepared from tert-butyl piperazine-1-carboxylate 13 and methyl 3-bromo-4-chlorobenzoate according to a similar procedure described for the synthesis of 18a: ¹H NMR (400 MHz, DMSO-d₆) δ7.66 (s, 1H), 7.58 (d, J=8.4 Hz, 1H), 7.51 (d, J=10.8 Hz, 1H), 3.04 (br, 8H), 2.12 (s, 6H) ; ESI MS m/z 335 [M+H]⁺; HPLC 98.0% (AUC), t_{R}=11.5 min.

### Example 7

3-(4-(3,5-Dimethyl-1H-pyrazol-4-yl)piperazin-1-yl)-2-fluorobenzoic Acid 18 g. Compound 18g was prepared from tert-butyl piperazine-1-carboxylate 13 and methyl 3-bromo-2-fluorobenzoate according to a similar procedure described for the synthesis of 18a: ¹H NMR (400 MHz, DMSO-d6) δ7.29 (m, 1H), 7.19 (m, 1H), 7.12 (t, J=9.6 Hz, 1H), 3.03 (br, 8H), 2.12 (s, 6H); ESI MS m/z 319 [M+H]⁺; HPLC 98.3% (AUC), t_{R}=10.3 min.

### Example 8

5-(4-(3,5-Dimethyl-1H-pyrazol-4-yl)piperazin-1-yl)-2-fluorobenzoic Acid 18h. Compound 18h was prepared from tert-butyl piperazine-l-carboxylate 13 and methyl 5-bromo-2-fluorobenzoate according to a similar procedure described for the synthesis of 18a: ¹H NMR (400 MHz, DMSO-d₆) δ7.29 (m, 1H), 7.19 (m, 1H), 7.12 (t, J 9.6 Hz, 1H), 3.13 (br, 4H), 2.99 (br, 4H), 2.10 (s, 6H) ; ESI MS m/z 319 [M+H]⁺; HPLC>99% (AUC), t_{R}=10.6 min.

### Example 9

3-(4-(3,5-Dimethyl-1H-pyrazol-4-yl)piperazin-1-yl)-5-fluorobenzoic Acid 18i. Compound 18i was prepared from tert-butyl piperazine-1-carboxylate 13 and methyl 3-bromo-5-fluorobenzoate according to a similar procedure described for the synthesis of 18a: ¹H NMR (400 MHz, DMSO-d₆) δ7.28 (s, 1H), 6.99 (m, 2H), 3.24 (m, 4H), 2,98 (m, 4H), 2.10 (s, 6H); ESI MS m/z 319 [M+H]⁺; HPLC 97.6% (AUC), t_{R}=11.3 min.

Example 10: 5-(4-(3,5-Dimethyl-1H-pyrazol-4-yl)piperazin-1-yl)-4-fluoro-2-methylbenzoic Acid 18j. Compound 18j was prepared from tert-butyl piperazine-1-carboxylate 13 and methyl 5-bromo-4-fluoro-2-methylbenzoate according to a similar procedure described for the synthesis of 18a: ¹H NMR (400 MHz, DMSO-d₆) δ7.52 (d, J=8.4 Hz, 1H), 7.13 (d, J=13.2 Hz, 1H), 3.04 (br, 8H), 2.45 (s, 1H), 2.13 (s, 6H); ESI MS m/z 333 [M+H]⁺; HPLC 98.7% (AUC), t_{R}=11.6 min.

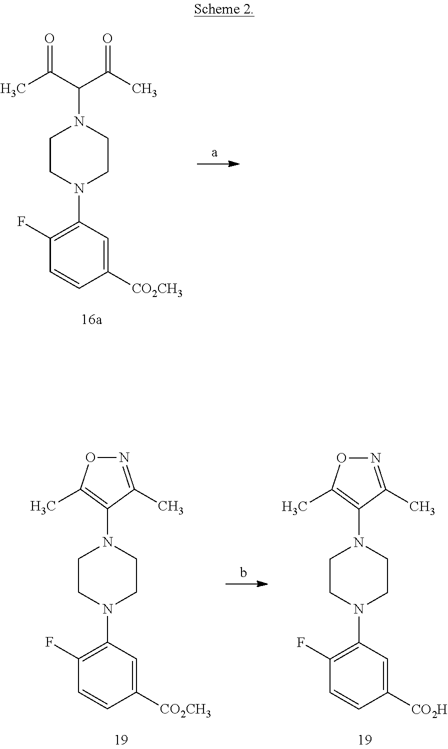 [SMILES: COC(=O)c1ccc(F)c(N2CCN(C(C(C)=O)C(C)=O)CC2)c1 ; COC(=O)c1ccc(F)c(N2CCN(c3c(C)noc3C)CC2)c1 ; Cc1noc(C)c1N1CCN(c2cc(C(=O)O)ccc2F)CC1]

Reagents and conditions: (a) NH₂ OH.HCl, CH₃ OH, rt, 16 h; (b) (i) LiOH, CH₃OH, THF, H₂O, rt, 16 h; (ii) neutralization to pH=7 with 2 N aqueous HCl.

### Example 11

3-(4-(3,5-Dimethylisoxazol-4-yl)piperazin-1-yl)-4-fluorobenzoic Acid 20. Step A: To a solution of methyl 3-(4-(2,4-dioxopentan-3-yl)piperazin-1-yl)-4-fluorobenzoate 16a (80.0 mg, 0.23 mmol) in CH₃OH (2 mL) was added NH₂ OH·HCl(32.0 mg, 0.47 mmol) and the resulting solution was stirred at rt for 16 h. The mixture was concentrated under reduced pressure and the resulting residue was chromatographed over silica gel (0-50% EtOAc in hexanes) to give methyl 3-(4-(3,5-dimethylisoxazol-4-yl)piperazin-1-171)-4-fluorobenzoate 19 as a brown solid (20.1 mg, 25%); ESI MS m/z 334 [M+H]⁺.

Step B: To a solution of methyl 3-(4-(3,5-dimethylisoxazol-4-yl)piperazin-1-171)-4-fluorobenzoate 19 (8.1 mg, 0.023 mmol) in CH₃OH (1 mL), THF (1 mL) and H₂O (0.5 mL) was added LiOH (2.7 mg, 0.11 mmol). The reaction mixture was stirred at rt for 16 h and then concentrated under reduced pressure. The aqueous layer was diluted with H₂O (15 mL) and neutralized to pH=7 with 2 N aqueous HCl. The aqueous mixture was extracted with EtOAc (3×10 mL) and the combined organic extracts were washed with brine, dried over Na₂SO₄, filtered, and concentrated under reduced pressure. The resulting residue was chromatographed over silica gel (0-10% CH₃OH in CH₂Cl₂) to give 3-(4-(3,5-dimethylisoxazol-4-yl)piperazin-1-yl)-4-fluorobenzoic acid 20 as a white solid (2.5 mg, 34%): ¹H NMR (400 MHz, DMSO-d₆) δ7.71-7.59 (m, 1H), 7.19-7.10 (m, 2H), 3.18-3.3.17 (m, 4H), 3.12-3.11 (m, 4H), 2.38 (s, 3H), 2.25 (s, 3H); ESI MS m/z 320 [M +H]⁺; HPLC 96.8% (AUC), t_{R}=13.7 min.

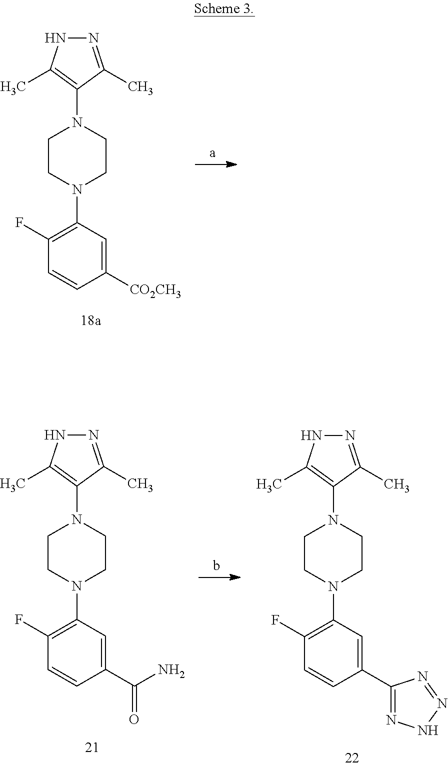 [SMILES: COC(=O)c1ccc(F)c(N2CCN(c3c(C)n[nH]c3C)CC2)c1 ; Cc1n[nH]c(C)c1N1CCN(c2cc(C(N)=O)ccc2F)CC1 ; Cc1n[nH]c(C)c1N1CCN(c2cc(-c3nn[nH]n3)ccc2F)CC1]

Reagents and conditions: (a) NH₄Cl, HBTU, i-Pr₂NEt, DMF, rt, 18 h; (b) NaN₃, tetrachlorosilane, CH₃CN, 80° C., 18 h.

### Example 12

3-(4-(3,5-Dimethyl-1H-pyrazol-4-yl)piperazin-1-yl)-4-fluorobenzamide 21. Step A: Step A: To a mixture of 3-(4-(3,5-dimethyl-1H-pyrazol-4-yl)piperazin-1-171)-4-fluorobenzoic acid 18a (0.100 g, 0.314 mmol), HBTU (0.178 g, 0.471 mmol), and i-Pr₂NEt (0.218 mL, 1.26 mmol) in DMF (4 mL) was added NH₄Cl (16.7 mg, 0.314 mmol). The resulting solution was stirred at rt for 18 h under an atmosphere of N₂. The mixture was diluted with H₂O (10 mL) and extracted with EtOAc (3×20 mL). The combined organic extracts were washed with H₂O (3×20 mL) and brine, dried over Na₂SO₄, filtered, and concentrated under reduced pressure. The resulting crude residue was chromatographed over silica gel (0% to 80% EtOAc in hexanes) to give 3-(4-(3,5-dimethyl-1H-pyrazol-4-yl)piperazin-1-yl)-4-fluorobenzamide 21 as a white solid (71.7 mg, 72%): ¹H NMR (400 MHz, DMSO-d₆) δ11.84 (br, 1H), 7.94 (bs, 1H), 7.54-7,48 (m, 2H), 7.31 (s, 1H), 7.14 (m, 1H), 3.06-3.01 (m, 8H), 2.10 (s, 6H); ESI MS m/z 318 [M+H]⁺; HPLC>99% (AUC), t_{R}=10.5 min.

### Example 13

1-(3,5-Dimethyl-1H-pyrazol-4-yl)-4-(2-fluoro-5-(2H-tetrazol-5-yl)phenyl)piperazine 22. Step A: A mixture of 3-(4-(3,5-dimethyl-1H-pyrazol-4-yl)piperazin-1-yl)-4-fluorobenzamide 21 (0.180 g, 0.526 mmol), NaN³ (0.142 g, 0.375 mmol), and tetrachlorosilane (98.5 mg, 0.579 mmol) in CH₃CN (4 mL) stirred at 80° C. for 18 h in a sealed vessel. The reaction mixture was allowed to cool to rt and diluted with saturated NaHCO₃ (5 mL). The aqueous mixture was extracted with CHCl₃ (3×50 mL) and the combined organic extracts were washed with brine (50 mL), dried over Na₂SO₄, filtered, and concentrated under reduced pressure. The resulting residue was chromatographed over silica gel (0% to 10% CH₃OH in CH₂Cl₂) to give 1-(3,5-dimethyl-1H-pyrazol-4-yl)-4-(2-fluoro-5-(2H-tetrazol-5-yl)phenyl)piperazine 22 as a white solid (60.3 mg, 30%): ¹H NMR (400 MHz, DMSO-d₆) δ7.69 (m, 1H), 7.67 (m, 1H), 7.36-7. (dd, J=4 Hz, J=8.4 Hz, 1H); 3.12 (m, 4H), 3.09 (m, 4H), 2.13 (s, 6H) ; ESI MS m/z 343 [M+H]⁺, HPLC>99% (AUC), t_{R}=11.1 min.

### Example 14

### Fluorescence Polarization TTR Tetramer Binding Assay.

Compound binding to TTR was assessed in the fluorescence polarization assay. The assay measured competitive displacement of the fluorescent probe, FITC-diclofenac, from TTR isolated from human plasma (Clabiochem-Millipore, cat. No. 52957). FITC-diclofenac was synthesized at LeadGen Labs, LLC. Each well contained 200 nM TTR and 100 nM FITC-diclofenac in the FP buffer (10 mM Tris-HCl pH 7.5, 150 mM NaCl, 0.01% CHAPS, 0.01% Prionex) along with test compounds. Nonspecific binding was determined in the presence of 500 μM unlabeled diclofenac (Sigma-Aldrich). Reactions with test compounds were incubated overnight at 4° C. and FP was measured on SpectramaxM5e plate reader (Molecular Devices).

### Example 15

### TTR Aggregation Assay

The ability of test compounds to prevent TTR aggregation was evaluated under the acidic conditions that favor TTR aggregation and fibril formation. A 2 μl solution of 167 μM human TTR (ACRO Biosystems #H5223) was incubated with 7 μl 50 mM sodium acetate (pH=4.0) (Sigma # S7545), 100 mM KCl (Sigma # S5405) in the presence or absence of 1 μl TTR inhibitor for 72 h at 37° C. At the end of the incubation, 3.5 μl 500 mM sodium phosphate (Sigma #S5136) buffer (pH=8.0) was added to each sample for neutralization and 0.6 μl 5% CHAPS (Sigma #C5070) as a detergent to prevent reassociation of protein. The cross-linking was performed by adding 1.5 μl 5% glutaraldehyde solution (Sigma# G6257). After 4 min, the reaction was stopped by the addition of 2.5 μl freshly made 5% NaBH₄. Samples were subjected to TTR western blotting with prealbumin antibodies (1:500; Dako #A0002). Band intensity for TTR monomer and TTR aggregates was quantified from scanned images of the blots.

### Example 16

In vitro binding of compounds to RBP4. Compound binding to RBP4 was assessed in the radiometric scintillation proximity (SPA) assay that was previously described (Cioffi, C. L. et al. 2014; Cioffi, C. L. et al. 2015; Cioffi, C. L. et al. 2020). The assay measured competitive displacement of radiolabeled [³H]-all-trans-retinol from native RBP4 purified from human urine (Fitzgerald, 30R-AR₀₂₂L). The protein was biotinylated using the EZ-link Sulfo-NHS-LC-Biotinylation kit from ThermoFisher (Cat #21335) as recommended by the manufacturer. Binding assays were implemented in a final volume of 100 μL in SPA buffer (1×PBR, pH 7.4, 1 mM EDTA, 0.1% BRA, 0.5% CHAPS). The assay reaction included a radioligand, 10 nM [³H]-all-trans-retinol (48.7 Ci/mmol; PerkinElmer, Waltham, MA), along with the 0.3 mg/well Streptavidin-PVT beads (PerkinElmer, RPNQ0006) and 50 nM biotinylated human RBP4. Unlabeled retinol (Sigma, cat # 95144) at 20 μM was added to control wells to assess a nonspecific binding. Radioactivity counts were measured using CHAMELEON plate reader (Hidex Oy, Turku, Finland) after 16 h of incubation at rt with mild shaking.

TABLE 1
TTR fluorescence polarization and RBP4 SPA
binding affinity data for select compounds.

| | TTR FP^{a} | RBP4 SPA^{b} |
|---|---|---|
| Compound | IC₅₀ (μM) ^{c} | IC₅₀ (μM) ^{c} |
| Tafamadis (3) | 0.41 | >30 |
| AG10 (4) | 0.16 | >30 |
| 18a | 0.22 | >3 |
| 18b | 0.78 | >30 |
| 18c | 0.30 | >3 |
| 18d | 0.21 | >3 |
| 18e | 0.26 | >3 |
| 18f | 0.20 | >3 |
| 18g | 0.55 | >3 |
| 18h | 1.44 | >3 |
| 18i | 0.62 | >3 |
| 18j | 0.60 | >3 |
| 20 | 5.3 | >3 |
| 21 | 0.67 | >3 |
| 22 | 0.26 | >3 |

^{a}IC₅₀ values for the fluorescence polarization (FP) assay obtained in the presence of a fixed, 25 μM concentration of fluorescein isothiocyanate (FITC)-coupled TTR FP probe.
^{b}IC₅₀ values for the SPA assay obtained in the presence of a fixed, 10 nM concentration of ³H-retinol.
^{c} For compounds tested multiple times (more than twice) the IC₅₀ data is represented as the mean ± standard deviation. For those compounds that were only tested twice, the IC₅₀ data is shown as the mean of two independent experiments and not as the mean ± standard deviation.

### Example 17

### Kinetic Solubility Assay

Kinetic aqueous solubility determination for compound 18a in PBS (pH 7.4) was conducted by Eurofins using UV detection (230 nm). Aqueous solubility (μM) was determined by comparing the peak area of the principal peak in a calibration standard (200 μM) containing organic solvent (methanol/water, 60/40, v/v) with the peak area of the corresponding peak in a buffer sample. In addition, chromatographic purity (%) was defined as the peak area of the principal peak relative to the total integrated peak area in the HPLC chromatogram of the calibration standard. A chromatogram of the calibration standard of each test compound, along with a UV/VIS spectrum with labeled absorbance maxima, was generated.

### Standards for the kinetic solubility study:

Metoprolol—192.6 μM

Rifampicin—200 μM

Ketoconazole—152.8 μM

Phenytoin—101.8 μM

Simvastatin—14.2 μM

Diethylstilbesterol—7.0 μM

Tamoxifen—1.9 μM

Example 18

### CYP450 Inhibition Assay

Inhibition potential (IC50 values) results for compound 18a against the human cytochrome P450 (CYP) isoforms 2C9, 2C19, 2D6, and 3A4.

Each recombinant human CYP isoform was tested with a standard positive and negative control, using fluorometric detection for measuring CYP activity. The measured IC₅₀ values for the respective standard inhibitors were all within expected ranges for each isoform (see below).

### IC₅₀ Concentrations of Standard CYP Inhibitors

CYP Inhibitor IC50 (μM):

2C9 Sulfaphenazole IC₅₀=3.4 μM

2C19 Tranylcypromine IC₅₀=2.8 μM

2D6 Quinidine IC₅₀=0.058 μM

3A4 Ketoconazole IC₅₀=0.0084 μM

Pre-formulated NADPH regenerating solutions, recombinant CYP isoforms 2C19 and 3A4 (Lot # 3007790 and 2276593 respectively), 3-[2-(N,N-diethyl-N-methylamino)ethyl]-7-methoxy-4-methylcoumarin (AMMC), 3-cyano-7-ethoxycoumarin (CEC) and 7-benzyloxy-4-trifluoromethylcoumarin (BFC) were obtained from Corning Life Sciences (Bedford, MA). Recombinant CYP isoform 2D6 (Lot # 49242) was obtained from Invitrogen (Carlsbad, CA). CYP isoform 2C9 (Lot #0446966-1) was obtained from Cayman Chemical (Ann Arbor, MI). 7-methoxy-4-trifluoromethylcoumarin (MFC), trans-2-phenylcyclopropylamine HCl (TCP), sulfaphenazole (SFZ), ketoconazole (KTZ) and quinidine (QDN) were obtained from Sigma (St. Louis, MO). All solvents and buffers were obtained from commercial sources and used without further purification.

### Methods

Test compound was prepared as a 10 mM stock solution in acetonitrile. Four human P450 isoforms cDNA-expressed in insect cell microsomes (CYP2C9, CYP2C19, CYP2D6, and CYP3A4) were tested for inhibition by test compound using fluorescence-based assays. Nine serial dilutions (concentrations from 0-100 μM) using each test compound stock solution were prepared in black microtiter plates, in duplicate. This dilution series was incubated at 37° C. with the individual CYP isoforms and a standard fluorogenic probe substrate for each respective isoform. The concentration of the probe substrate added was at or near the Km value for each CYP isoform. Reaction mixtures contained potassium phosphate buffer, pH 7.4 and the NADPH-regenerating system. The final reaction volume was 0.20 mL and the reaction was terminated with 75 μL of stop solution (0.5 M Tris base in acetonitrile) after the appropriate incubation time (15-45 minutes). Fluorescence measurements were made at the appropriate excitation and emission wavelengths. Duplicate control wells with no test compound, duplicate blank wells containing stop solution prior to adding isoform, and a dilution series in duplicate containing a standard inhibitor for each isoform were also conducted. IC₅₀ values were calculated using a non-linear regression of the data using the four-parameter logistic model (dose response equation) fit with XLFit 5.2 from IDBS Software (Emeryville, CA), supported by linear interpolation of data points at concentrations indicating inhibition levels approximately 50% of the uninhibited rate.

### Example 19

### Plasma Protein Binding Assay

Plasma protein binding (PPB) for compounds determination for compound 18a in PBS (pH 7.4) was conducted by Eurofins using equilibrium dialysis of plasma with HPLC-UV/Vis detection.

### Mean Plasma Protein Binding of Control Propranolol in Human, Rat (Sprague Dawley), Mouse (CD-1), and Dog (Beagle) Plasma

The peak areas of the test compound in the buffer and test samples were used to calculate percent binding and recovery according to the following formulas:

$$Protein binding ( \% ) = \frac{{Area}_{p} - {Area}_{b}}{{Area}_{p}} * 100$$

$$Recovery ( \% ) = \frac{{Area}_{p} + {Area}_{b}}{{Area}_{c}} * 100$$

### Where:

Areaₚ=Peak area of analyte in protein matrix
Area_{b}=Peak area of analyte in buffer
Area_{c}=Peak area of analyte in control sample

### Example 20

### Metabolic Stability

### Metabolic Stability in Microsomes

The results of metabolic stability determinations for novel compounds and testosterone (positive control) were conducted in the presence of human, rat, mouse, and monkey liver microsomes. Values shown are percent of parent remaining after a 30 minute incubation. All measurements were done in duplicate. Assay results for testosterone were within an acceptable range.

### Metabolic Clearance in Microsomes

Mixed-gender human liver microsomes (Lot# 1710084), male Sprague-Dawley rat liver microsomes (Lot# 1610290), male CD-1 mouse liver microsomes (Lot# 1710069), and male Cynomolgous liver microsomes (Lot# 1510193) were purchased from XenoTech. The reaction mixture, minus NADPH, was prepared as described below. The test article was added into the reaction mixture at a final concentration of 1 μM. The control compound, testosterone, was run simultaneously with the test article in a separate reaction. An aliquot of the reaction mixture (without cofactor) was equilibrated in a shaking water bath at 37° C. for 3 minutes. The reaction was initiated by the addition of cofactor, and the mixture was incubated in a shaking water bath at 37° C. Aliquots (100 μL) were withdrawn at 0, 10, 20, 30, and 60 minutes. Test article and testosterone samples were immediately combined with 400 μL of ice-cold 50/50 acetonitrile (ACN)/H₂O containing 0.1% formic acid and internal standard to terminate the reaction. The samples were then mixed and centrifuged to precipitate proteins. All samples were assayed by LC-MS/MS using electrospray ionization. The peak area response ratio (PARR) to internal standard was compared to the PARR at time 0 to determine the percent remaining at each time point. Half-lives were calculated using GraphPad software, fitting to a single-phase exponential decay equation.

TABLE 2
In vitro ADME profile for 18a.

| | Microsomal CL [symbol] | | | | Liver Microsomal Stability | | | | CYP Inhibition | | | | | |
|---|---|---|---|---|---|---|---|---|---|---|---|---|---|---|
| | (μL/min/mg) [symbol] | | | | (% remaining at 30 min)^{c} | | | | (% Inhibition at 10 μm) | hERG [symbol] | PPARγ | % PPB [symbol] | | |
| Solubility [symbol] | H | R | M | cyno | HLM | RLM | MLM | cyno LM | 2C9, 2C19, 2D6, 3A4 | (IC₅₀) | (EC₅₀) | H | R | M |

| | | | | | | | | | | | |
|---|---|---|---|---|---|---|---|---|---|---|---|
| 184.5 μM | <0.0231 | 93 | 98 | 102 | 94 | 2C9 - 10.4% | >30 μM | >100 μM | 83 | 97 | 93 |

2C19 - 3.1%
2D6 - 8.1%
3A4 - (−)2.2%

^{a}Kinetic solubility measured in PBS (pH = 7.4).
^{b}Microsomal intrinsic clearance (CLᵢₙₜ); H = human; R = rat; M = mouse; cyno = cynomolgus monkey.
^{c}Liver microsomal metabolic stability, % of parent drug remaining after a 30 minute incubation in the presence of the microsomes; HLM = human liver microsomes; RLM = rat liver microsomes; MLM = mouse liver microsomea; cyno LM = cynomolgus monkey liver microsomes.
^{d}CiPA hERG QPatch Assay; compounds were tested (n = 2) in a five-point concentration-response study.
^{e}% PPB = plasma protein binding; H = human, R = rat, M = mouse. [symbol]
indicates data missing or illegible when filed

### Example 21

### In Vivo PK Assay

### Mouse PK Study Information and Data

Drug naive adult male CD-1 mice were administered a single dose administration of the test article by intravenous (IV) or oral gavage (P0) dose routes.

### Testing Facility and Test Site: Absorption Systems, LLC, 436

Creamery Way, Suite 600, Exton, PA 19341-2556

### Test Article and Vehicle Information

IV dosing vehicles: 3% DMA/45% PEG300/12% ethanol/40% sterile water
PO dosing vehicle: 2% Tween 80 in 0.9% saline
Dose formulation: The dose formulation was prepared by the step-wise addition (in the order listed) of the individual components of the vehicle to a weighed quantity of test compound in a volume that yielded the desired final concentration.

Each formulation was prepared by mixing a weighed quantity of test compound with the appropriate volume of vehicle.

Dosing Solution Analysis: The dosing solutions were analyzed by LC-MS/MS. The dosing solutions were diluted into mouse blood and analyzed in triplicate. All concentrations are expressed as mg/mL of the free base. The nominal dosing level was used in all calculations for Group 1.

### Test System

Species and strain: mouse; male CD-1
Mean weight: 0.034 kg for the IV arm; 0.027 kg for PO arm
Number: 3 animals total (same three animals used for each dosing group (Group 1 (IV) and Group 2 (PO))

COMPLIANCE: This non-clinical study followed established practices and standard operating procedures of Absorption Systems as well as the study protocol. This study was exploratory in nature and was not conducted in accordance with the principles set forth in the United States Food and Drug Administration (FDA) Good Laboratory Practice (GLP) Regulations, 21 Code of Federal regulations (CFR) Part 58. The report is archived in a validated scientific data management system. Electronic signatures comply with the regulation 21 CFR Part 11.

### Experimental Design

Blood was collected from mice at pre-dose and at 5, 15 and 30 min, and 1, 2, 4, 8, 12, 24 and 48 h post-dose. Hemolyzed blood samples were extracted by protein precipitation using acetonitrile.

Following protein extraction with acetonitrile, compound levels were measured by LC-MS/MS. Pharmacokinetic parameters were calculated from the time course of the blood concentrations. Pharmacokinetic parameters were determined with Phoenix WinNonlin (v8.0) software using a non-compartmental model. The maximum blood concentrations (Co) after IV dosing were estimated by extrapolation of the first two time points back to t=0. The maximum blood concentration (Cmax) and the time to reach maximum blood concentration (tmax) after PO dosing were observed from the data. The area under the time concentration curve (AUC) was calculated using the linear trapezoidal rule with calculation to the last quantifiable data point, and with extrapolation to infinity if applicable. Blood half-life (t ½) was calculated from 0.693/slope of the terminal elimination phase. Mean residence time, MRT, was calculated by dividing the area under the moment curve (AUMC) by the AUC. Clearance (CL) was calculated from dose/AUC. Steady-state volume of distribution (Vss) was calculated from CL*MRT. Bioavailability was determined by dividing the individual dose normalized PO AUC∞ values by the average dose-normalized IV AUC∞ value. Any samples below the limit of quantitation (1.00 ng/mL) were treated as zero for pharmacokinetic data analysis.

TABLE 3
In Vivo PK Data for Analogue 18a Following IV and PO Administration in CD-1 mice.

| | | C [symbol] ^{a} | CL^{b} | t_{1/2}^{c} | Vₛₛ^{d} | AUCₗₐₛₜ^{e} | AUC_{INF}^{f} | |
|---|---|---|---|---|---|---|---|---|
| Route | Dose | (ng/ml) | (L/hr/kg) | (h) | (L/kg) | (hr · ng/mL) | (hr · ng/mL) | % Fⁱ |
| IV | 2 mg/kg | 1622 | 0.354 | 5.08 | 2.18 | 6214 | 6242 | NA |
| | | (267) | (0.139) | (0.814) | (0.585) | (2321) | (2303) | |
| | | C [symbol] ^{g} | | t_{1/2}^{c} | Vₛₛ^{d} | AUCₗₐₛₜ^{e} | AUC_{INF}^{f} | |
| Route | Dose | (ng/ml) | Tₘₐₓ^{h} (h) | (h) | (mL/kg) | (hr · ng/mL) | (hr · ng/mL) | % Fⁱ |
| PO | 5 mg/kg | 1563 | 0.42 | 5.38 | NA | 16040 | 16073 | 103 |
| | | (115) | (0.14) | (0.624) | | (2778) | (2783) | (17.8) |

[symbol]

indicates data missing or illegible when filed

Data are represented as the mean with standard deviation in parentheses (mean (SD)). Dosing groups consisted of three drug naive adult male CD-1 mice. IV administration: Test article was administered at the 2 mg/kg dose; test article vehicle=3% DMA/45% PEG300/12% Ethanol/40% Sterile water; PO administration: test article was administered at the 5 mg/kg dose, vehicle=2% Tween 80 in 0.9% saline. ^{a}Observed initial concentration of compound in blood at time zero. ^{b}Total body clearance. ^{c}Apparent half-life of the terminal phase of elimination of compound from blood. ^{d}Volume of distribution at steady state. ^{e}Area under the blood concentration versus time curve from 0 to the last time point that compound was quantifiable in blood. ^{f}Area under the blood concentration versus time curve from 0 to infinity. ^{g}Maximum observed concentration of compound in blood. ^{h}Time of maximum observed concentration of compound in blood. ⁱBioavailability; F=(AUC_{INFpo}×Doseᵢᵥ)÷AUC_{INFiv}×Doseₚₒ).

### Example 22

### Serum RBP4 Measurement Assay

Blood samples were collected from a tail vein. Whole blood was drawn into a centrifuge tube and was allowed to clot at room temperature for 30 minutes followed by centrifugation at 2000 g for 15 minutes at 48° C. to collect serum. Aliquots of plasma samples collected in the mouse pharmacokinetic study were analyzed for the RBP4 concentration using the RBP4 (mouse/rat) dual ELISA kit (AdipoGen, San Diego, CA) following the manufacturer's instructions. Whole blood was drawn into a centrifuge tube and was allowed to clot at room temperature for 30 min followed by centrifugation at 2000 g for 15 min at +4° C to collect serum. Mouse serum RBP4 (produced predominantly in the liver) was measured using the RBP4 (mouse/rat) dual ELISA kit (AdipoGen, San Diego, CA; catalog number AG-45A-0012YTP-KI01).

### Example 23

### Efficacy in the Abca4^{-/-} Model

Long-term 12-week dosing of 18a2HCl formulated into a chow was conducted in Abca4^{-/-} mice. The age-matched control group of Abca4^{-/-} mice was kept on a standard Picolab 5053 chow without the compound. The age-matched reference group of wild type mice was used for defining the basal level of A2E in mice in the absence of the Abca4 ablation; these Abca4^{+/+} mice were kept on a standard Picolab 5053 chow. Blood samples for assessing the serum levels of RBP4 were collected from compound-treated and control chow-treated Abca4^{-/-} mice at pre-dose, at Week 4, and at the end of the compound treatment at Week 8. Significant serum RBP4 reduction was documented in compound-treated mice (FIG. 7). Following the 8 weeks of dosing, the eyecups of treated and untreated Abca4^{-/-} mice as well as the eyecups of the reference Abca4^{+/+} mice were collected for the quantitative A2E analysis which was conducted by HPLC. Significant A2E reduction was seen in compound-treated Abca4^{-/ -} mice in comparison to untreated Abca4^{-/-} mice (FIG. 8). A 98.5% reduction in A2E content induced by 18a·2HCl brought the A2E levels below the levels seen in the untreated wild type animals (FIG. 8).

In addition to biochemical characterization of the A2E content by HPLC, we conducted the assessment of A2E fluorescence in retinal sections from untreated Abca4^{+/+} mice (FIG. 9A), untreated Abca4^{-/-} mice (FIG. 9B), and 18a·2HCl-treated Abca4^{-/-} mice (FIG. 9C). Genetic ablation of the Abca4 gene induced a significant increase in A2E autofluorescence in the Abca4^{-/-} mice (FIG. 9B) in comparison to wild type control (FIG. 9A). Treatment with 18a·2HCl significantly reduced the intensity of A2E autofluorescence (FIG. 9C) consistent with robust inhibition of bisretinoid synthesis.

Animal Care and Use Statement: All procedures are in compliance with the U.S. Department of Agriculture's (USDA) Animal Welfare Act (9 CFR Parts 1, 2, and 3); the Guide for the Care and Use of Laboratory Animals, Institute of Laboratory Animal Resources, National Academy Press, Washington, D.C., 1996; and the National Institutes of Health, Office of Laboratory Animal Welfare. Whenever possible, procedures in this study are designed to avoid or minimize discomfort, distress, and pain to animals.

### Discussion

A structure-based drug design effort was used to identify a novel class of TTR tetramer kinetic stabilizers that are capable of reducing circulating levels of RBP4 using clinically investigated 4 (FIG. 2) as a lead. 4 was selected as a benchmark scaffold from which to develop a novel series of ligands as 1) 4 has been reported to effectively bind at both WT-TTR and the pro-amyloidogenic V122I-TTR variant, and 2) 4 is also reported to be more potent and selective for stabilizing TTR tetramers in buffer and human serum than 3, despite both compounds exhibiting similar TTR binding affinities (K_{d} for 4=4.8±1.9 nM; K_{d} for 3=4.4±1.3 nM) (Miller, M. et al. 2018; Penchala, S. C. et al. 2013).

Compounds were initially evaluated in two assays designed to measure (1) compound binding affinity to unliganded TTR tetramers (fluorescence polarization assay, FP) and (2) compound binding affinity to non-TTR associated RBP4 (scintillation proximity assay, SPA). The results are shown in Table 1. Compound 18a was approximately 2-fold more potent at TTR (FP IC₅₀=220 nM) compared to benchmark 3 (FP IC₅₀=410 nM) and was comparable in potency compared to benchmark 4 (FP IC₅₀=160 nM) (Table 1). 18a also demonstrated good selectivity over RBP4 as it was found to be inactive in the in vitro RBP4 scintillation proximity (SPA) assay (RBP4 SPA IC₅₀>3 μM), which is used to measure binding potency of all-trans-retinol competitive ligands at RBP4 (Table 1).

The ability of TTR ligands to act as kinetic stabilizers of TTR tetramers was assessed in vitro using a low pH-induced TTR aggregation SDS-PAGE assay (Coelho, T. et al. 2016; Cruz, M. W. 2019). A 72 h incubation of TTR tetramers at pH=4.0 initiates its dissociation into monomeric intermediates that misassemble and oligomerize into amyloid fibrils and other high molecular weight forms (Lamb, Y. N. & Deeks, E. D. 2019). The ability of compound 18a to act as a kinetic stabilizer of TTR tetramers was assessed by its ability to suppress low pH-mediated TTR aggregate formation using a previously published protocol (Park, J. et al. 2020; Cioffi, C. L. et al. 2020). FDA-approved 3 was used as a positive control in the aggregation experiments. As shown in FIG. 5A, following 72 h of incubation of TTR tetramers with DMSO at pH=4 at 37° C., the amount of high molecular weight forms of TTR are greatly increased in comparison to the DMSO control incubated for 72 h at pH=7.5.

Consistent with its ability to act as a kinetic TTR stabilizer, treatment with compound 18a (50 μM) as well as with 3 (50 μM), significantly inhibited the formation of high molecular weight forms of TTR (FIG. 5A). Higher intensities of the TTR monomer and dimer bands in samples treated with 18a and 3 when compared to DMSO correlated with a corresponding reduction in TTR aggregates induced by 18a and 3. Quantification of Western blot band intensities established a 3.1-fold reduction in the amount of aggregates in the presence of 18a while a 3.0-fold reduction was induced by 3 (FIG. 5B). A significant increase in the dimer band intensities and appreciable increase in the intensity of TTR monomer bands conferred by 18a and 3 were associated with inhibition of the low pH-induced TTR aggregate formation. (FIG. 5, C and D). These results showed that 18a can act as a TTR tetramer kinetic stabilizer.

Compound 18a exhibited excellent kinetic solubility in phosphate buffered saline (PBS) (pH 7.4) and the observed microsomal stability and CL±values suggest very low predicted hepatic clearance across multiple species (Table 2). The % plasma protein binding (PPB) data indicates low fraction unbound in human, rat and mouse (Table 2). In addition, 18a lacked limiting inhibitory activity in a standard CYP panel, at the hERG channel, or at the nuclear peroxisome proliferator-activated receptor-gamma (PPAR_{γ}) receptor (Table 2).

Compound 18a showed favorable plasma clearance (0.354 L/hr/kg) and a half-life of 5.08 h following administration of a single dose (2 mg/kg IV) to CD-1 male mice (Table 3). The compound was well absorbed and slowly eliminated from plasma following oral administration of a single dose (5 mg/kg) with an observed C max of 1563 ng/ml and corresponding Tₘₐₓ at 0.42 h (Table 3). Compound 18a demonstrated good overall exposure (AUC_{INF} was 16073 hr·ng/mL) and excellent oral bioavailability (% F=103%).

After a single 25 mg/kg oral dose of 18a, a maximal 66% reduction in serum RBP4 was observed 2 h after administration (FIG. 6A). There was no effect of compound dosing on serum TTR levels (data not shown). The dynamics of the in vivo serum RBP4 reduction demonstrated a good correlation between the presence of 18a in circulation after oral dosing (FIG. 6B) and a reduction in serum RBP4 (FIG. 6A). The maximum RBP4 reduction observed at the 1 and 2 h timepoints correlated very well with rapid oral absorption that resulted in high concentrations of 18a in the blood at these time points (FIG. 6A,B). Similarly, the low level of 18a in the blood at 24 h corresponds well with serum RBP4 levels (FIG. 6A,B). This data shows that there is a very good PK/PD relationship between 18a exposure and serum RBP4 lowering activity in mice.

A direct correlation has been previously established between serum RBP4 lowering induced by different classes of selective RBP4 antagonists and bisretinoid-lowering efficacy in the Abca4^{-/-} mouse model of Stargardt disease (Radu, R. A. et al. 2005; Racz, B. et al. 2018; Dobri, N. et al. 2013). Based on the very good in vivo RBP4 lowering activity exhibited by 18a, it is expected that this compound may be efficacious in suppressing the formation of cytotoxic lipofuscin bisretinoids in the retina, which justifies evaluation of selective TTR ligands as a class of potential therapeutics for the treatment of Stargardt disease, dry AMD and other conditions characterized by enhanced accumulation of lipofuscin in the retina.

These results show a novel class of TTR tetramer kinetic stabilizers that selectively bind to TTR tetramers. As such, these compounds have application for the treatment of ATTR-CM, ATTR-PN, FAP, FAC or SSA and other ATTR diseases. These ligands are also able to reduce circulating levels of RBP4 in vivo. Therefore, in addition to diseases characterized by ATTR, these compounds may also be efficacious in suppressing the formation of cytotoxic lipofuscin bisretinoids in the retina while also preventing possible TTR amyloid fibril formation. Therefore, these selective TTR tetramer ligands may also have use as therapeutics for Stargardt disease, dry AMD and other conditions characterized by enhanced accumulation of lipofuscin in the retina, especially in patients who are also prone to ATTR comorbidities such as sporadic SSA or hereditary TTR amyloidosis.

### REFERENCES

Alhamadsheh, M. M. et al. Sci. Transl. Med. 3, 97ra81 (2011).

Berk, J. L. et al. JAMA 310, 2658-2667 (2013).

Bulawa, C. E. et al. Proc. Natl. Acad. Sci. U.S.A. 109, 9629-9634 (2012).

Cioffi, C. L. et al. J. Med. Chem. 57, 7731-7757 (2014).

Cioffi, C.L. et al. J. Med. Chem. 58, 5863-5888 (2015).

Cioffi, C.L. et al. J. Med. Chem. 62, 5470-5500 (2019).

Cioffi, C.L. et al. J. Med. Chem. 63, 11054-11084 (2020).

Coelho, T. et al. J. Neurol. 260, 2802-2814 (2013).

Coelho, T. et al. Neurol. Ther. 5, 1-25 (2016).

Cruz, M. W. Clin. Auton. Res. 29 (Suppl 1), 19-24 (2019).

Damrauer, S. M. et al. JAMA 322, 2191-2202 (2019).

Dobri, N. et al. Invest. Ophth. Vis. Sci. 54, 85-95 (2013).

Gales, L. et al. Biochem. J. 388, 615-621 (2005).

Giao, T. et al. Int. J. Mol. Sci. 21, 2075 (2020).

Gimeno, A. et al. J. Med. Chem. 60, 5749-5758 (2017).

Hoy, S. M. Drugs 78, 1625-1631 (2018).

Hyung, S. J. et al. ACS. Chem. Biol. 5, 1137-1146 (2010).

Jesus, C. S. et al. Int. J. Mol. Sci. 17, 1428 (2016).

Jiang, X. et al. Proc. Natl. Acad. Sci. U.S.A 98, 14943-14948(2001).

Judge, D. P. et al. J. Am. Coll. Cardiol. 74, 285-295 (2019).

Kamata, M. et al. J. Gene. Med. 11, 103-111 (2009).

Kanai, M. et al. J. Clin. Invest. 47, 2025-2044 (1968).

Kassem, N. A. et al. Am. J. Physiol. Regul. Integr. Comp. Physiol. 291, R₁₃₁₀-R₁₃₁₅ (2006).

Kawaguchi, R. et al. Membranes (Basel) 5, 425-453 (2015).

Lamb, Y. N. & Deeks, E. D. Drugs 79, 863-874 (2019).

Leach, B. I. et al. Biochemistry 57, 4421-4430 (2018).

Mathew, V. & Wang, A. K. Drug. Des. Devel. Ther. 13, 1515-1525 (2019).

Maurer, M .S. et al. N. Engl. J. Med. 379, 1007-1016 (2018).

Miller, M. et al. J. Med. Chem. 61, 7862-7876 (2018).

Park, G. Y. et al. Int. J. Mol. Sci. 20, 2982 (2019).

Park, J. et al. Ann. Pharmacother. 54, 470-477 (2020).

Penchala, S.C. et al. Proc. Natl. Acad. Sci. U.S.A. 110, 9992-9997 (2013).

Racz, B. et al. J. Biol. Chem. 293, 11574-11588 (2018).

Racz, B. et al. PLoS One 15, e0228291 (2020).

Radu, R. A. et al. Invest. Ophth. Vis. Sci., 46, 4393-4401 (2005).

Ruberg, F. L. et al. J. Am. Coll. Cardiol. 73, 2872-2891 (2019).

Sant'Anna, R. et al. Nat. Commun. 7, 10787 (2016).

Sousa, M. M. et al. Am. J. Pathol. 161, 1935-1948 (2002).

Sun, X. et al. Proc. Natl. Acad. Sci. U S A 115, E6201-E6208 (2018).

Vieira, M. & Saraiva, M. J. Biomol. Concepts 5, 45-54 (2014).

Waddington-Cruz, M. et al. Orphanet. J. Rare Dis. 14, 34 (2019).

Yamamoto, H. & Yokochi, T. ESC Heart Fail. 6, 1128-1139 (2019).

What is claimed is:

1. A compound having the structure: 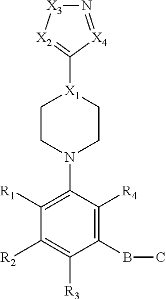
wherein
X₁ is N or CR₅,
wherein R₅ is H, OH, halogen or alkyl;
X₂, X₃ and X₄ are each independently NH, N, S, O or CR₆,
wherein each R₆ is independently H, OH, halogen, alkyl, alkenyl, alkynyl, cycloalkyl, cycloalkylalkyl, haloalkyl, —O-(alkyl), —S-(alkyl), —NH₂, —NH-(alkyl), —N(alkyl)₂ or —CO₂H; R₁, R₂, R₃ and R₄ are each independently —H, —F, —Cl, —Br, —I, —NO₂, —CN, —CF₃, —CF₂H, —OCF₃, -(alkyl), -(haloalkyl), -(alkenyl), -(alkynyl), -(aryl), -(heteroaryl), -(cycloalkyl), -(cycloalkylalkyl), -(heteroalkyl), heterocycle, heterocycloalkyl, -(alkylheteroalkyl), -(alkylaryl), —OH, —OAc, —O-(alkyl), —O-(alkenyl), —O-(alkynyl), —O-(aryl), —O-(heteroaryl), —SH, —S-(alkyl), —S-(alkenyl), —S-(alkynyl), —S-(aryl), —S-(heteroaryl), —NH₂, —NH-(alkyl), —NH-(alkenyl), —NH-(alkynyl), —NH-(aryl), —NH-(heteroaryl), —C(O)R₇, —S(O)R₇, —SO₂R₇, —NHSO₂R₇, —OC(O)R₇, —SC(O) R₇, —NHC (O)R₇ or —NHC(S)R₇, wherein R₇ is, H, -(alkyl), —OH, —O(alkyl), —NH₂, —NH(alkyl) or —N(alkyl)₂;
B is absent or present, and when present is 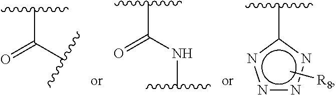
wherein R₈ is H, OH, halogen, alkyl, cycloalkyl, cycloalkylalkyl, —O-(alkyl), —S-(alkyl), —NH₂, —NH-(alkyl), —N(alkyl)₂ or —CO₂H;
and
C is H, substituted or unsubstituted monocycle, bicycle, heteromonocycle, heterobicycle, aryl, heteroaryl, alkyl, cycloalkyl, cycloalkylalkyl, CO₂H, COOR₉, OH, OR₉, NH₂, NHR₉, NR₉R₁₀, SO₂R₁₁, CH₂NHR₉, CH₂NR₉R₁₀ or CH₂COOR₉, wherein R₉ and R₁₀ are each independently H, alkyl, cycloalkyl, —C(O)-alkyl, —C(O)-cycloalkyl, —C(O)OH, —C(O)—O-alkyl, —C(O)—O-cycloalkyl, —C(O)NH₂, —C(O)NH(alkyl), —C(O)NH(cycloalkyl), —C(O)N(alkyl)₂, —CH₂NH(alkyl), —CH₂COOH, —SO₂CH₃, —OH, —O(alkyl), —NH₂, —NH(alkyl) or —N(alkyl)₂,
wherein R₁₁ is alkyl, haloalkyl, cycloalkyl, aryl, heteroaryl, NH₂, NH(alkyl), NH(cycloalkyl), NH(heterocycle), NH(aryl), NH(heteroaryl) or NHCOR₁₂,
wherein R₁₂ is alkyl, haloalkyl, cycloalkyl, heterocycle, aryl or heteroaryl,
or a pharmaceutically acceptable salt thereof.

2. The compound of claim 1, wherein
X₁ is N or CR₅,
wherein R₅ is H, OH, halogen or alkyl;
X₂, X₃ and X₄ are each independently NH, N, S, O or CR₆,
wherein each R₆ is independently H, OH, halogen, alkyl, alkenyl, alkynyl, cycloalkyl, cycloalkylalkyl, haloalkyl, —O-(alkyl), —S-(alkyl), —NH₂, —NH-(alkyl), —N(alkyl)₂ or —CO₂H;
R₁, R₂, R₃ and R₄ are each independently —H, —F, —Cl, —Br, —I, —NO₂, —CN, —CF₃, —CF₂H, —OCF₃, -(alkyl), -(haloalkyl), -(alkenyl), -(alkynyl), -(aryl), -(heteroaryl), -(cycloalkyl), -(cycloalkylalkyl), -(heteroalkyl), heterocycle, heterocycloalkyl, -(alkylheteroalkyl), -(alkylaryl), —OH, —OAc, —O-(alkyl), —O-(alkenyl), —O-(alkynyl), —O-(aryl), —O-(heteroaryl), —SH, —S-(alkyl), —S-(alkenyl), —S-(alkynyl), —S-(aryl), —S-(heteroaryl), —NH₂, —NH-(alkyl), —NH-(alkenyl), —NH-(alkynyl), —NH-(aryl) or —NH-(heteroaryl);
B is absent or present, and when present is 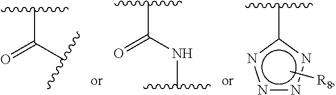
wherein R₈ is H, OH halogen, alkyl, cycloalkyl, cycloalkylalkyl, —O-(alkyl), —S-(alkyl), —NH₂, —NH-(alkyl), —N(alkyl)₂ or —CO₂H;
and
C is H, substituted or unsubstituted monocycle, bicycle, heteromonocycle, heterobicycle, aryl, heteroaryl, alkyl, cycloalkyl, cycloalkylalkyl, CO₂H, COOR₉, OH, OR₉, NH₂, NHR₉, NR₉R₁₀, SO₂R₁₁, CH₂NHR₉, CH₂NR₉R₁₀ or CH₂COOR₉,
wherein R₉ and R₁₀ are each independently H, alkyl, cycloalkyl, —C(O)-alkyl, —C(O)-cycloalkyl, —C(O)OH, —C(O)—O-alkyl, —C(O)—O-cycloalkyl, —C(O)NH₂, —C(O)NH(alkyl), —C(O)NH(cycloalkyl), —C(O)N(alkyl)₂, —CH₂NH(alkyl), —CH₂COOH, —SO₂CH₃, —OH, —O(alkyl), —NH₂, —NH(alkyl) or —N(alkyl)₂,
wherein R₁₁ is alkyl, haloalkyl, cycloalkyl, aryl, heteroaryl, NH₂, NH(alkyl), NH(cycloalkyl), NH(heterocycle), NH(aryl), NH(heteroaryl) or NHCOR₁₂,
wherein R₁₂ is alkyl, haloalkyl, cycloalkyl, heterocycle, aryl or heteroaryl,
or a pharmaceutically acceptable salt thereof.

3. The compound of claim 2, wherein
X₁ is N;
X₂, X₃ and X₄ are each independently NH, N, S, O or CR₆,
wherein each R₆ is independently H, halogen, alkyl, alkenyl, alkynyl, haloalkyl, —O-(alkyl), —S-(alkyl), —NH₂, —NH-(alkyl), —N(alkyl)₂ or —CO₂H;
R₁, R₂, R₃ and R₄ are each independently —H, —F, —Cl, —Br, —I, —CN, —CF₃, —CF₂H, —OCF₃, -(alkyl), -(alkenyl), -(alkynyl), -(aryl), -(heteroaryl), -(cycloalkyl), -(cycloalkylalkyl), -(heteroalkyl), heterocycle, heterocycloalkyl, -(alkylheteroalkyl), -(alkylaryl), —OH, —OAc, —O-(alkyl), —O-(alkenyl), —O-(alkynyl), —O-(aryl), —O-(heteroaryl), —NH₂, —NH-(alkyl), —NH-(alkenyl), —NH-(alkynyl), —NH-(aryl) or —NH-(heteroaryl); and
B-C is -CO₂H, —CONH₂ or 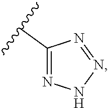 [SMILES: *c1nn[nH]n1]
or a pharmaceutically acceptable salt thereof.

4. The compound of claim 3 having the structure: 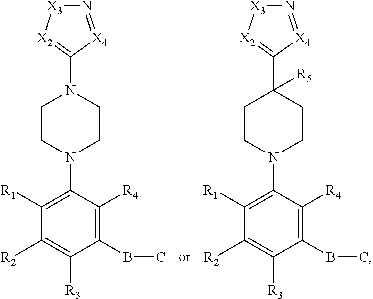
or a pharmaceutically acceptable salt thereof.

5. (canceled)

6. The compound of claim 4,
wherein
X₃ is NH, and X₂ and X₄ are CR₆, or
X₃ is O, and X₂ and X₄ are CR₆, or
X₃ is S, and X₂ and X₄ are CR₆, or
wherein R₆ is H, OH, alkyl, alkenyl, alkynyl, haloalkyl, —O-(alkyl), —S-(alkyl), —NH₂, —NH-(alkyl), —N(alkyl)₂ or —CO₂H, or
wherein R₆ is alkyl, or
wherein R₆ is methyl or —CF₃, or
wherein B-C is —CO₂H, —CONH₂ or
wherein B-C is —CO₂H, or 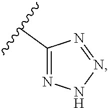 [SMILES: *c1nn[nH]n1]
or
wherein R₁, R₂, R₃ and R₄ are each independently —H, —F, —Cl, —Br, —I, —NO₂, —CN, —CF₃, —CF₂H, —OCF₃, -(alkyl), -(haloalkyl), -(alkenyl), -(alkynyl), —OH, —OAc, —O-(alkyl), —S-(alkyl), or
wherein R₁, R₂, R₃ and R₄ are each independently H, F, Cl, CH₃, CF₃ or OCH_{3 .}

7-13. (canceled)

14. The compound of claim 6, wherein
R₁ is H, F, Cl, CH₃, CF₃ or OCH₃, or
R₂ is H, F, Cl, CH₃, CF₃ or OCH₃, or
R₃ is H, F, Cl, CH₃, CF₃ or OCH₃, or
R₄ is H, F, Cl, CH₃, CF₃ or OCH₃, or
wherein R₁ is H, F, Cl, CH₃, CF₃ or OCH₃, and R₂, R₃ and R₄ are each H, or
R₁ is F, Cl, CH₃, CF₃ or OCH₃, R₃ is CH₃, and R₂ and R₄ are each H, or
R₁ is F and R₂, R₃ and R₄ are each independently H, F, Cl, CH₃,
CF₃ or OCH₃, or
R₁ is F and R₂, R₃ and R₄ are each H, or
R₁ is Cl and R₂, R₃ and R₄ are each independently H, F, Cl, CH₃, CF₃ or OCH₃, or
R₁ is Cl and R₂, R₃ and R₄ are each H, or
wherein R₁ is F or Cl, R₂, R₃ and R₄ are each H, and B-C is —CO₂H, or
R₁ is F or Cl, R₂, R₃ and R₄ are each H, and B-C is −CONH₂, or
R₁ is F or Cl, R₂, R₃ and R₄ are each H, and B-C is 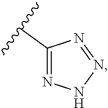 [SMILES: *c1nn[nH]n1]
or
wherein B-C is —CO₂H, —CONH₂ or

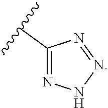 [SMILES: *c1nn[nH]n1]

15-17. (canceled)

18. The compound of claim 6, having the structure: 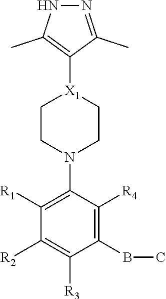
or a pharmaceutically acceptable salt thereof,
wherein X₁ is N or CR₅,
wherein B-C is -CO₂H, —CONH₂, or

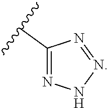 [SMILES: *c1nn[nH]n1]

19-20. (canceled)

21. The compound claim 18, having the structure:

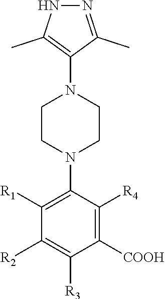

22. The compound of claim 18, wherein R₁, R₂, R₃, and R₄ are each independently H, F, Cl, CH₃, CF₃ or OCH₃, or
wherein R₁ is H, F, Cl, CH₃, CF₃ or OCH₃, or
R₂ is H, F, Cl, CH₃, CF₃ or OCH₃, or
R₃ is H, F, Cl, CH₃, CF₃ or OCH₃, or
R₄ is H, F, Cl, CH₃, CF₃ or OCH₃, or
wherein R₁ is H, F, Cl, CH₃, CF₃ or OCH₃ and R₂, R₃ and R₄ are each H, or R₁ is F, Cl, CH₃, CF₃ or OCH₃, R₃ is CH₃, and R₂ and R₄ are each H, or R₁ is F, and R₂, R₃ and R₄ are each H, or R₁ is Cl, and R₂, R₃ and R₄ are each H.

23-27. (canceled)

28. The compound of claim 1, wherein the compound has the structure: 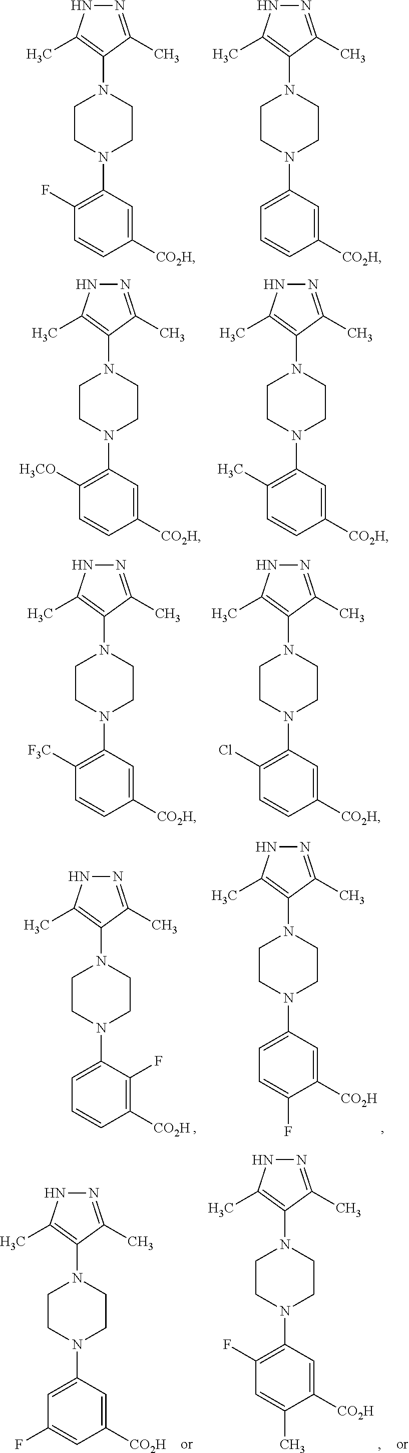 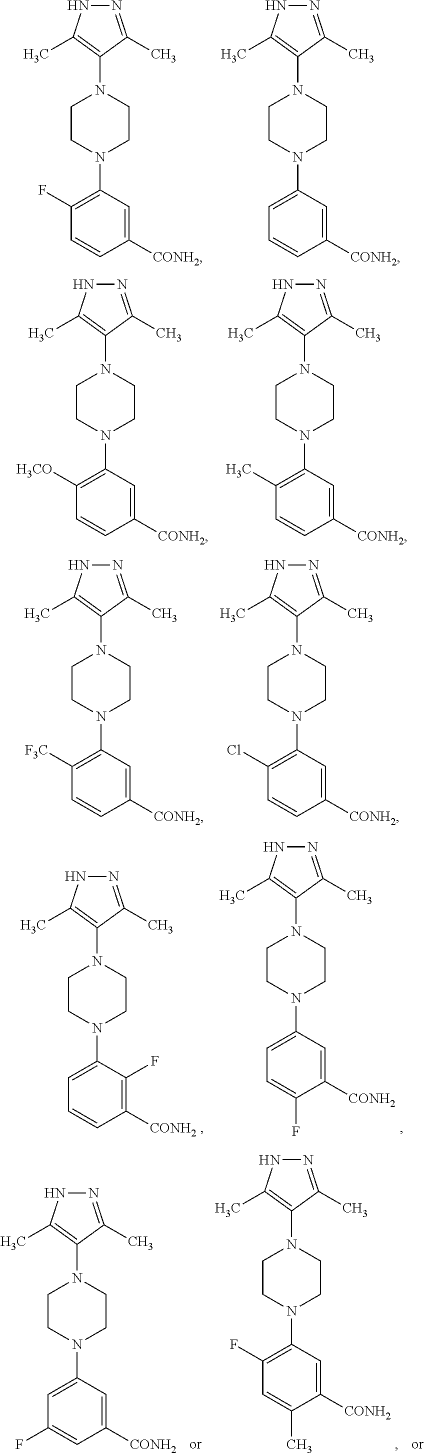 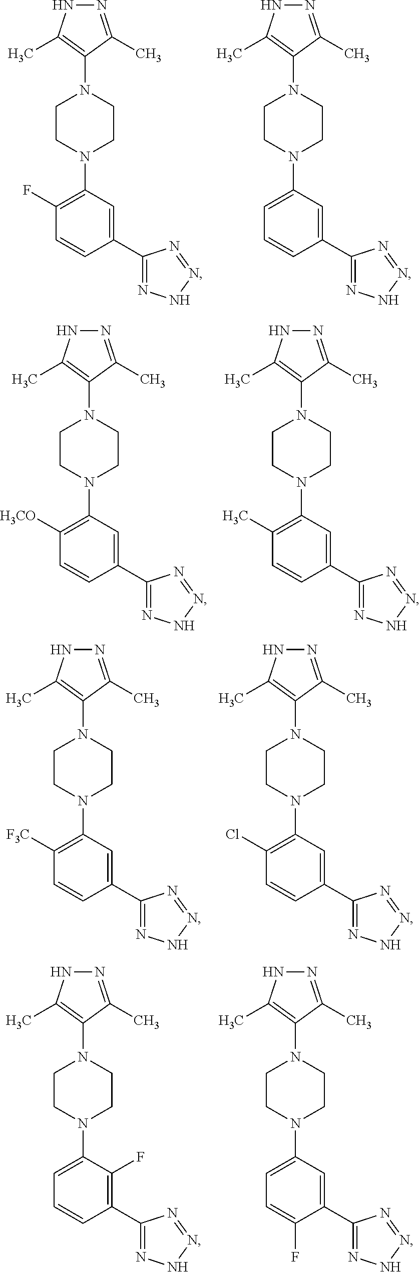 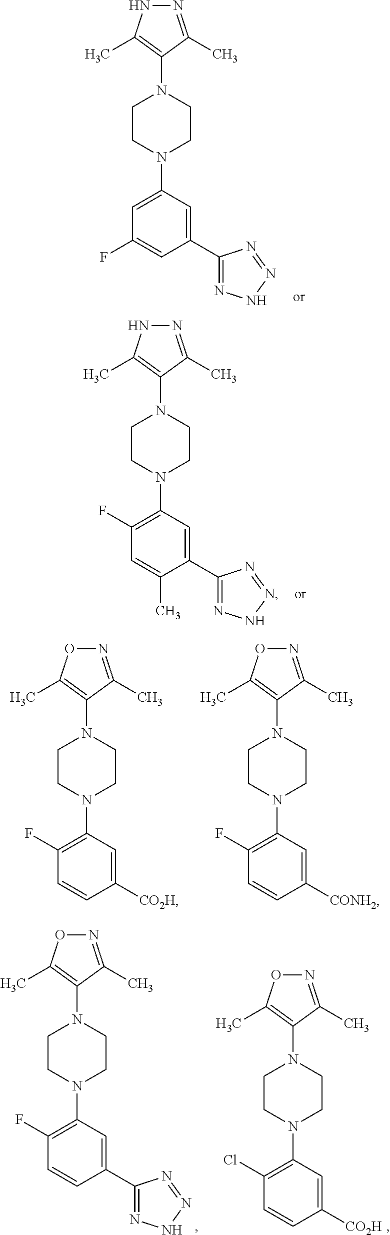 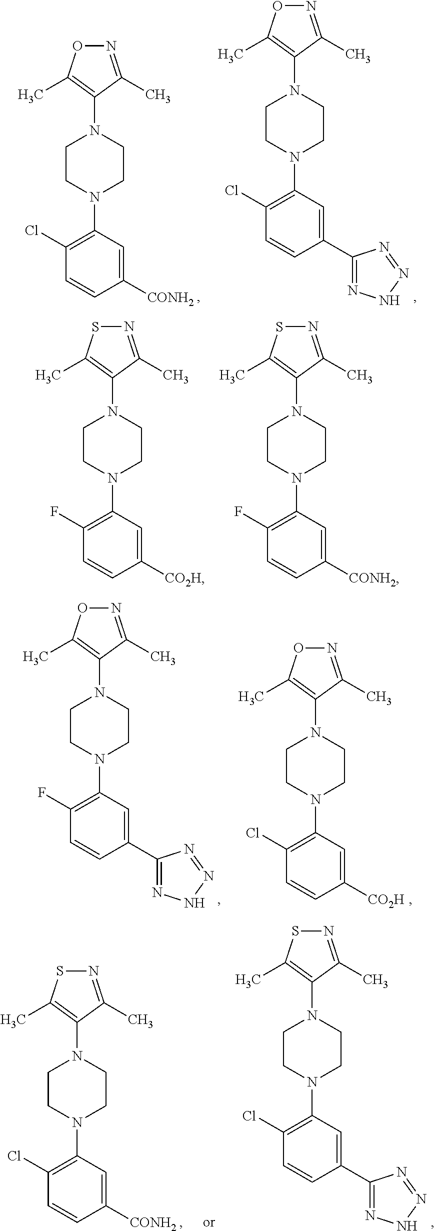
or a pharmaceutically acceptable salt of the compound.

29. The compound of claim 1, wherein the compound has the structure: 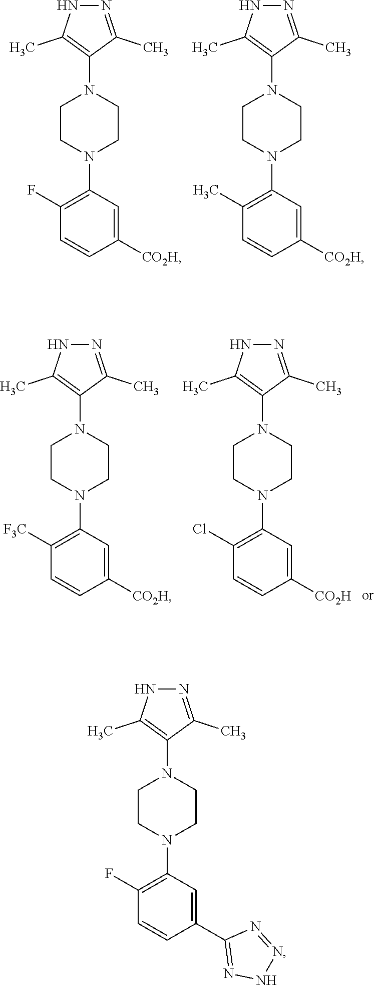
or a pharmaceutically acceptable salt of the compound.

30. A pharmaceutical composition comprising the compound of claim 28 and a pharmaceutically acceptable carrier.

31. A method for stabilizing TTR tetramers in a mammal comprising administering to the mammal an amount of a composition of claim 30 effective to stabilize TTR tetramers, or
of preventing TTR aggregate formation or preventing formation of high molecular weight aggregates in a mammal comprising administering to the mammal an amount of the composition of claim 30 effective to prevent TTR aggregate formation or prevent formation of high molecular weight aggregates, or
for treating a TTR amyloidosis (ATTR) disease, in a mammal afflicted therewith comprising administering to the mammal an effective amount of a composition of claim 30, or
for treating a disease characterized by excessive lipofuscin accumulation in the retina, in a mammal afflicted therewith comprising administering to the mammal an effective amount of a composition of claim 30,
wherein the disease is further characterized by bisretinoid-mediated macular degeneration,
wherein the disease characterized by excessive lipofuscin accumulation in the retina is Age-Related Macular Degeneration, dry (atrophic) Age-Related Macular Degeneration, Stargardt Disease, Best disease, adult vitelliform maculopathy or Stargardt-like macular dystrophy, or
for treating a disease characterized by a TTR amyloidosis (ATTR) disease, or by excessive lipofuscin accumulation in the retina, or both a TTR amyloidosis (ATTR) disease and a disease characterized by excessive lipofuscin, in a mammal afflicted therewith comprising administering to the mammal an effective amount of a composition of claim 30.

32-33. (canceled)

34. The method of claim 31, wherein the method is further effective to stabilize TTR tetramers in the mammal,
wherein the TTR amyloidosis (ATTR) disease is peripheral polyneuropathy (ATTR-PN), TTR amyloid cardiomyopathy (ATTR-CM), late-onset familial amyloid polyneuropathy (FAP), familial amyloid cardiomyopathy (FAC) or senile systemic amyloidosis (SSA), or
wherein the TTR amyloidosis (ATTR) disease is characterized by deposition of amyloid aggregates.

35-38. (canceled)

39. The method of claim 31, wherein the amount of the composition is effective to lower the serum concentration of RBP4 in the mammal, or wherein the amount of the composition is effective to lower the retinal concentration of a bisretinoid in lipofuscin in the mammal,
wherein the bisretinoid is A2E, isoA2E, A2-DHP-PE or atRAL di-PE.

40-42. (canceled)

43. The method of claim 3, wherein the amount of the composition is effective to stabilize TTR tetramers in the mammal, or
wherein the amount of the composition is effective to prevent TTR aggregate formation or prevent formation of high molecular weight aggregates.

44. (canceled)

45. The method of claim 43, wherein the amount of the composition is effective to lower the serum concentration of RBP4 in the mammal, or wherein the amount of the composition is effective to lower the retinal concentration of a bisretinoid in lipofuscin in the mammal.

46. The method of claim 3, wherein the amount of the compound is effective to stabilize TTR tetramers in the mammal and to lower the serum concentration of RBP4 in the mammal, or wherein the amount of the composition is effective to prevent TTR aggregate formation or prevent formation of high molecular weight aggregates in the mammal and to lower the serum concentration of RBP4 in the mammal, or wherein the amount of the composition is effective to stabilize TTR tetramers in the mammal and to lower the retinal concentration of a bisretinoid in lipofuscin in the mammal, or wherein the amount of the composition is effective to prevent TTR aggregate formation or prevent formation of high molecular weight aggregates in the mammal and to lower the retinal concentration of a bisretinoid in lipofuscin in the mammal, or
wherein the TTR amyloidosis (ATTR) disease is peripheral polyneuropathy (ATTR-PN), TTR amyloid cardiomyopathy (ATTR-CM), late-onset familial amyloid polyneuropathy (FAP), familial amyloid cardiomyopathy (FAC) or senile systemic amyloidosis (SSA), or
wherein the TTR amyloidosis (ATTR) disease is characterized by deposition of amyloid aggregates, or
wherein the disease is further characterized by bisretinoid-mediated macular degeneration.

47-49. (canceled)

50. The method of claim 46, wherein the amount of the composition is effective to lower the serum concentration of RBP4 in the mammal, or wherein the amount of the composition is effective to lower the retinal concentration of a bisretinoid in lipofuscin in the mammal,
wherein the bisretinoid is A2E, isoA2E, A2-DHP-PE or atRAL di-PE.

51. (canceled)

52. The method of claim 50, wherein the disease characterized by excessive lipofuscin accumulation in the retina is Age-Related Macular Degeneration, dry (atrophic) Age-Related Macular Degeneration, Stargardt Disease, Best disease, adult vitelliform maculopathy or Stargardt-like macular dystrophy.
